(19) United States
(12) Patent Application Publication
(10) Pub. No.: US 2023/0120141 A1
(43) Pub. Date: Apr. 20, 2023

(54) Polyethylene Glycol Conjugates of Polyethyleneimine and Their Use in Gene Therapy

(71) Applicant: CHARLES RIVER LABORATORIES, INC., Wilmington, MA (US)

(72) Inventor: Aslam ANSARI, Wilmington, MA (US)

(21) Appl. No.: 17/938,727

(22) Filed: Oct. 7, 2022

Related U.S. Application Data
(60) Provisional application No. 63/253,687, filed on Oct. 8, 2021.

(51) Int. Cl. A61K 48/00; C08L 79/02; C12N 15/86

(52) U.S. Cl. CPC A61K 48/0041; C08L 79/02; C12N 15/86; C12N 2750/14143; C12N 2770/00043

(57) ABSTRACT

The present invention is directed to improved methods for conjugating polyethylene glycol to polyethyleneimine, and to the use of such polyethylene glycol—polyethyleneimine conjugates to improve the efficiency with which viral and non-viral nucleic acid vectors transfect cells to provide gene therapy, and to improve the efficiency of producing viral particles that comprise therapeutic polynucleotides for use in gene therapy.

### CROSS REFERENCE TO RELATED APPLICATIONS

The present application claims the benefit of U.S. Provisional Application No. 63/253,687, filed on Oct. 8, 2021, the entire contents of which are incorporated herein by reference.

### FIELD OF THE INVENTION

The present invention is directed to improved methods for conjugating polyethylene glycol to polyethyleneimine, and to the use of such polyethylene glycol—polyethyleneimine conjugates to improve the efficiency with which viral and non-viral nucleic acid vectors transfect cells to provide gene therapy, and to improve the efficiency of producing viral particles that comprise therapeutic polynucleotides for use in gene therapy.

### BACKGROUND OF THE INVENTION

### I. Gene Therapy

Traditional pharmaceuticals are synthetic chemicals that are designed to alter the body's chemistry so as to provide transient relief by reducing the symptoms of disease without specifically targeting the overall cause of the disease (Goswami, R. et al. (2019) “Gene Therapy Leaves a Vicious Cycle,” Front. Oncol. 9:297:1-25). The deficiencies of such pharmaceuticals are partially addressed by protein-based therapeutics (such as signaling proteins, gene-editing enzymes, growth factors, hormones, blood factors, antibodies, and protein antigens (Lagassé, H. A. et al. (2017) “Recent Advances In (Therapeutic Protein) Drug Development,” F1000Research 6:113:1-17).

However, protein-based therapies are associated with a number of drawbacks, such as complex production and manufacturing, low solubility, short circulating half-lives, in vivo physicochemical instability, limited ability to penetrate into target tissue, and adverse patient reactions, such as immune system reactivity, inflammation, and fever (Lagassé, H. A. et al. (2017) “Recent Advances In (Therapeutic Protein) Drug Development,” F1000Research 6:113:1-17; Spicer, C. D. et al. (2018) “Peptide And Protein Nanoparticle Conjugates: Versatile Platforms For Biomedical Applications,” Chem. Soc. Rev. 47(10):3574-3620).

Gene therapy has the potential to address such issues. Gene therapy involves the use of nucleic acid molecules as pharmaceutical agents to treat pathogenic or genetic diseases. The nucleic acid molecules mediate their effect by being transcribed and/or translated after being delivered into recipient target cells, or by integrating into the chromosomes of such cells. The use of such a treatment approach surmounts many of the drawbacks of protein-based therapeutics (Hanna, E. et al. (2017) “Gene Therapies Development. Slow Progress And Promising Prospect,” J. Mark. Access Health Policy 5:1265293:1-9; Wirth, T. et al. (2013) “History Of Gene Therapy,” Gene 525:162-169; Goswami, R. et al. (2019) “Gene Therapy Leaves a Vicious Cycle,” Front. Oncol. 9:297:1-25; Spicer, C. D. et al. (2018) “Peptide And Protein Nanoparticle Conjugates: Versatile Platforms For Biomedical Applications,” Chem. Soc. Rev. 47(10):3574-3620). Gene therapy is particularly suitable for the treatment of inheritable or acquired diseases.

Transfection of nucleic acids into cells or tissue, however, is not simple and is dependent on overcoming a number of barriers. For example, the employed vector must be able to (1) stabilize the nucleic acid and prevent it from being degraded; (2) reach desired target cells; (3) disrupt the endosomal membrane of such target cells, and (4) deliver the nucleic acid molecule into the nucleus or cytoplasm of the target cells (PCT Publn. WO 2010/088927 A1). A primary concern in gene therapy is achieving efficient gene delivery. Gene delivery systems are designed to protect and control the location of a gene within the body by affecting the distribution and access of a gene expression system to the target cell, and/or recognition by a cell-surface receptor, followed by intracellular trafficking and nuclear translocation (Friedmann, T. (1999) “The Development of Human Gene Therapy,” Cold Spring Harbor Laboratory Press, San Diego).

There are generally two types of delivery vehicles used in gene therapy: viral and non-viral vectors, both of which present specific advantages and disadvantages (Thomas, T. J. et al. (2019) “Biodegradable Polymers for Gene Delivery,” Molecules 24(20):3744:1-24; Guo, X. et al. (2012) “Recent Advances In Nonviral Vectors For Gene Delivery,” Accounts Chem. Res. 45(7):971-919; Wasala, N. B. et al. (2011) “The Evolution Of Heart Gene Delivery Vectors,” J. Gene Med. 13(10):557-565; Pannier, A. K. et al. (2004) “Controlled Release Systems For DNA Delivery,”. Mol. Ther. 10(1):19-26). As discussed below, currently employed viral vectors include retroviral vectors, adenoviral vectors and adeno-associated viral vectors. Viral vectors are highly effective in achieving high efficiency for both gene delivery and expression, and exhibit stable long-term expression of a foreign gene when the recombinant DNA is integrated into the chromosomal DNA (Milone, M. C. et al. (2018) “Clinical Use Of Lentiviral Vectors,” Leukemia 32:1529-1541; Naso, M. et al. (2017) “Adeno-Associated Virus (AAV) As A Vector for Gene Therapy,” BioDrugs 31:317-334). Some limitations of virally mediated gene delivery include limited DNA carrying capacity, toxicity, potential replication, immunogenicity, cancer formation and high cost. In particular, viral vectors are typically produced by transfecting host cells with plasmids that comprise the genes and sequences needed to produce viral particles that comprise the polynucleotide that is to be delivered. Thus, the use of viral vectors is dependent upon the use of non-viral transfection methods and non-viral vectors.

### II. Polymer-Mediated, Non-Viral Gene Transfer

Non-viral gene delivery systems, in particular, are based on genetic material being encompassed by or complexed to particles by electrostatic interactions between the negatively charged phosphate backbone of DNA and cationic particles, including, for example, polymers, lipids, or peptides (Erbacher, P. et al. (1999) “Gene Transfer With Synthetic Virus-Like Particles Via The Integrin-Mediated Endocytosis Pathway,” Gene Ther. 6(1):138-145). It has been suggested that complexes formed between a nucleic acid and a lipid attach to a cell surface, then pass into the cell by endocytosis. The complex is localized within a vesicle or endosome and the nucleic acid is released into the cytoplasm. Eventually, the nucleic acid migrates into the nucleus, where a gene encoded by the nucleic acid may be expressed. DNA is transcribed into RNA and then translated into protein (PCT Publn. WO 2018/035435 A1). Non-viral vectors have attracted great interest, as they are relatively simple to prepare, stable and easy to modify and relatively safe, as compared to viral vectors (Zu, H. et al. (2021) “Non-viral Vectors in Gene Therapy: Recent Development, Challenges, and Prospects,” AAPS J. 23(4):78:1-12; Guo, X. et al. (2012) “Recent Advances In Nonviral Vectors For Gene Delivery,” Accounts Chem. Res. 45(7):971-919; Goswami, R. et al. (2019) “Gene Therapy Leaves a Vicious Cycle,” Front. Oncol. 9:297:1-25). Unfortunately, non-viral vector systems have been associated with lower transfection efficiency, and have required additional effort, for example, the use of cationic polymers, for their optimization (see, e.g., Sung, Y. K. et al. (2020) “Recent Advances In Polymeric Drug Delivery Systems,” Biomater. Res. 24:12:1-12; U.S. Pat. Nos. 6,077,663; 6,274,322; 8,003,734; 8,258,235; 10,155,780; 10,968,240; US Patent Publn. US 2006/0147376 A1; US 2012/0083037 A1; US 2019/0290779 A1; PCT Publns. WO 2008/058457A1; WO 2021/023798 A1 and WO 2021/023796 A1).

Cationic polymers that are used in conjunction with non-viral vectors include: poly(L-Lysine) (PLL) (EP 1503802 B1; Wu, G. Y. (1987) “Receptor-Mediated In Vitro Gene Transformation By A Soluble DNA Carrier System,” J. Biol. Chem. 262(10):4429-4432; Wu, G. Y. et al. (1988) “Receptor-Mediated Gene Delivery And Expression in vivo,” J. Biol. Chem. 263(29):14621-14624; Mislick, K. A. et al. (1995) “Transfection Of Folate-Polylysine DNA Complexes: Evidence For Lysosomal Delivery,” Bioconjug Chem. 1995 September-October; 6(5):512-515; Wagner, E. et al. (1990) “Transferrin-Polycation Conjugates As Carriers For DNA Uptake Into Cells,” Proc. Natl. Acad. Sci. (U.S.A.) 87(9):3410-3414), polyethyleneimine (PEI) (Boussif, O. et al. (1995) “A Versatile Vector For Gene And Oligonucleotide Transfer Into Cells In Culture And in vivo: Polyethylenimine,” Proc. Natl. Acad. Sci. (U.S.A.) 92(16):7297-7301; Neuberg, P. et al. (2014) “Recent Developments In Nucleic Acid Delivery With Polyethylenimines,” Adv. Genet. 88:263-288; Baker, A. et al. (1997) “Polyethylenimine (PEI) Is A Simple, Inexpensive And Effective Reagent For Condensing And Linking Plasmid DNA To Adenovirus For Gene Delivery,” Gene Ther 4:773-782; Thomas, T. J. et al. (2019) “Biodegradable Polymers for Gene Delivery,” Molecules 24(20):3744:1-24), chitosan (Bonferoni, M. C. et al. (2020) “Chitosan Nanoparticles for Therapy and Theranostics of Hepatocellular Carcinoma (HCC) and Liver-Targeting,” Nanomaterials (Basel, Switzerland) 10(5):870:1-19; Thomas, T. J. et al. (2019) “Biodegradable Polymers for Gene Delivery,” Molecules 24(20):3744:1-24), PAMAM dendrimers (Tarach, P. et al. (2021) “Recent Advances in Preclinical Research Using PAMAM Dendrimers for Cancer Gene Therapy,” Int. J. Mol. Sci. 22(6):2912:1-30; de Araújo, R. V. et al. (2018) “New Advances in General Biomedical Applications of PAMAM Dendrimers,” Molecules 23(11):2849:1-27), and poly(2-dimethylamino)ethyl methacrylate (pDMAEMA) (Bhattarai, S. R. et al. (2010) “Enhanced Gene And siRNA Delivery By Polycation-Modified Mesoporous Silica Nanoparticles Loaded With Chloroquine,” Pharm. Res. 27:2556-2568).

Polyethyleneimine (PEI) is an example of a cationic polymer capable of carrying gene in vitro and in vivo into various cell lines and tissues. PEI may be obtained commercially (Sigma Aldrich; Polysciences), or may be synthesized (Weyts, K. F. et al. (1988) “New Synthesis Of Linear Polyethyleneimine,” Polymer Bulletin 19:13-19; Gosselin, M. A. et al. (2001) “Efficient Gene Transfer Using Reversibly Cross-Linked Low Molecular Weight Polyethylenimine,” Bioconjugate Chem. 12:232-245; Lynn, D. A. et al. (2001) “Accelerated Discovery of Synthetic Transfection Vectors: Parallel Synthesis and Screening of a Degradable Polymer Library,” J. Am. Chem. Soc. 123:8155-8156). Branched PEI can be synthesized by the ring-opening polymerization of aziridine (Zhuk, D. S. et al. (1965) “Advances In The Chemistry Of Polyethyleneimine (Polyaziridine),” Russ. Chem. Rev. 34(7):515-527). Linear PEI can be synthesized by post-modification of poly(2-oxazolines) (Tanaka, R. et al. (1983) “High Molecular Weight Linear Polyethylenimine And Poly(N-Methylethylenimine),” Macromolecules 16(6):849-853), or N-substituted polyaziridines (Weyts, K. F. et al. (1988) “New Synthesis Of Linear Polyethyleneimine,” Polymer Bulletin 19:13-19). Linear PEI has been synthesized by the hydrolysis of poly(2-ethyl-2-oxazoline) (Brissault, B. et al. (2003) “Synthesis of Linear Polyethyleneimine Derivatives for DNA Transfection” Bioconjugate Chem. 14(3):581-587). Several PEI transfection agents have been made commercially available, including ExGen500 (ThermoFisher; Ferrari, S. et al. (1997) “Exgen 500 Is An Efficient Vector For Gene Delivery To Lung Epithelial Cells in vitro and in vivo,” Gene Ther. 4:1100-1106) and jetPEI (Polyplus Transfection; Kasai, H. et al. (2019) “Efficient siRNA Delivery And Gene Silencing Using A Lipopolypeptide Hybrid Vector-Mediated By A Caveolae-Mediated And Temperature-Dependent Endocytic Pathway,” J. Nanobiotechnol. 17(1):11).

PEI promotes efficient gene transfer without the need for endosomolytic or targeting agents (Boussif, O. et al. (1995) “A Versatile Vector For Gene And Oligonucleotide Transfer Into Cells In Culture And in vivo: Polyethylenimine,” Proc. Natl. Acad. Sci. (U.S.A.) 92(16):7297-7301). Positively charged PEI polyplexes are endocytosed by cells, and PEI is also believed to facilitate endosomal escape due to its high density of secondary amines and tertiary amines. Unfortunately, higher molecular weight PEI has also been reported to be toxic to cells, which severely limits PEI's potential as a gene delivery tool in applications to human patients (see, e.g., EP 1503802 B1).

Thus, a need exists for a PEI polymer that is capable of exhibiting efficient gene transfer with decreased or no toxicity. The present invention is directed to this and other goals.

### SUMMARY OF THE INVENTION

The present invention is directed to improved methods for conjugating polyethylene glycol to polyethyleneimine, and to the use of such polyethylene glycol—polyethyleneimine conjugates to improve the efficiency with which viral and non-viral nucleic acid vectors transfect cells to provide gene therapy, and to improve the efficiency of producing viral particles that comprise therapeutic polynucleotides for use in gene therapy.

In detail, the invention provides a polyethylene glycol conjugate of polyethyleneimine comprising the Formula (III):

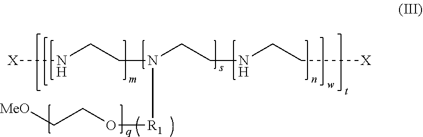

wherein: R₁ is a carbon-containing, hydroxyl-comprising group that may comprise, for example, one, two, three, or more than three, carbon atoms, and that is preferably not an ethyleneimine (—CH₂—CH₂—NH—) group;
each X is independently amine, methyl or methoxy, or an alternative terminating atom or chemical group;
m and n are independently zero or an integer;
s, w and t are independently an integer equal to or greater than 1;
m, n, and s may be the same, or may vary independently, for each w; and
q is an integer equal to or greater than 1, and may vary independently for each s.

The invention further provides the embodiment of such polyethylene glycol conjugate of polyethyleneimine (III), wherein R₁ is —CH₂—CH(OH)—CH₂—, and the polyethylene glycol conjugate of polyethyleneimine (III) is:

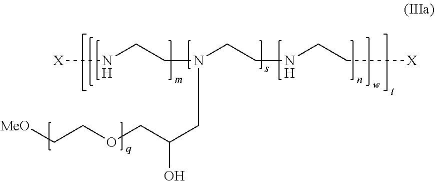

The invention further provides the embodiment of such polyethylene glycol conjugate of polyethyleneimine (III), wherein the conjugate comprises from 1 to approximately 2325 ethyleneimine monomer substituents.

The invention further provides the embodiment of such polyethylene glycol conjugate of polyethyleneimine (III), wherein the conjugate comprises from 1 to approximately 2272 ethylene glycol substituents.

The invention further provides the embodiment of such polyethylene glycol conjugate of polyethyleneimine (III) wherein:
(A) from about 2% to about 100% of the ethyleneimine monomer substituents thereof are conjugated to PEG;
(B) from about 5% to about 80% of the ethyleneimine monomer substituents thereof are conjugated to PEG;
(C) from about 10% to about 30% of the ethyleneimine monomer substituents thereof are conjugated to PEG; or
(D) about 10% of the ethyleneimine monomer substituents thereof are conjugated to PEG.

The invention further provides a method of producing such the polyethylene glycol conjugate of polyethyleneimine (III) of claim 1, which comprises:
(A) reacting polyethyleneimine of Formula (I)

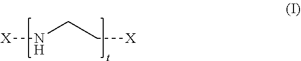

with polyethylene glycol of Formula (II)

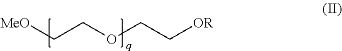

wherein R is an activated leaving group that reacts with a secondary amine N of (I) to form R₁;
   wherein R₁ and X are as defined above; in MeOH at neutral or basic pH.
(B) acidifying the reaction to thereby cause the polyethylene glycol conjugate of polyethyleneimine (III) to become insoluble; and
(C) recovering the polyethylene glycol conjugate of polyethyleneimine (III) from the insoluble fraction of the reaction.

The invention further provides the embodiment of such method wherein the polyethylene glycol (II) is polyethylene glycol-2-(methoxy)oxirane, and the polyethylene glycol conjugate of polyethyleneimine (III) is:

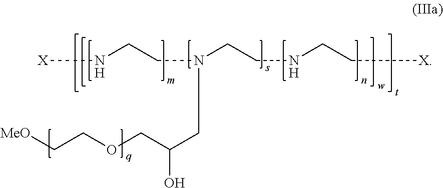

The invention further provides a composition comprising such polyethylene glycol conjugate of polyethyleneimine (III) and a polynucleotide molecule.

The invention further provides the embodiment of such composition wherein the composition is suitable for transfecting the polynucleotide into a recipient cell, wherein the presence of the polyethylene glycol conjugate of polyethyleneimine (III) enhances the efficiency of transfection or the viability of transfected cells relative to the efficiency of transfection or the viability of transfected cells when transfected in the absence of the polyethylene glycol conjugate of polyethyleneimine (III).

The invention further provides the embodiment of such composition wherein the efficiency of transfection or viability in the presence of the polyethylene glycol conjugate of polyethyleneimine (III) is at least 50% greater than the efficiency of transfection or the viability of such transfected cells when transfected in the absence of the polyethylene glycol conjugate of polyethyleneimine (III).

The invention further provides a composition comprising the polyethylene glycol conjugate of polyethyleneimine (IIIa):

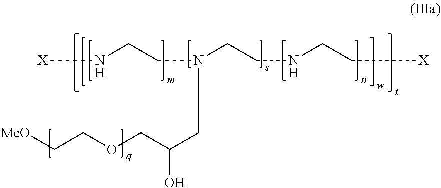

and a polynucleotide molecule.

The invention further provides the embodiment of such composition wherein the composition is suitable for transfecting the polynucleotide into a recipient cell, wherein the presence of the polyethylene glycol conjugate of polyethyleneimine (III) enhances the efficiency of transfection or the viability of transfected cells relative to the efficiency of transfection or the viability of transfected cells when transfected in the absence of the polyethylene glycol conjugate of polyethyleneimine (III).

The invention further provides the embodiment of such composition wherein the efficiency of transfection or viability in the presence of the polyethylene glycol conjugate of polyethyleneimine (III) is at least 50% greater than the efficiency of transfection or the viability of such transfected cells when transfected in the absence of the polyethylene glycol conjugate of polyethyleneimine (III).

The invention further provides the embodiment of such composition wherein the polynucleotide molecule comprises a non-viral vector.

The invention further provides the embodiment of such composition wherein the non-viral vector is a plasmid vector.

The invention further provides the embodiment of such composition wherein the polynucleotide molecule comprises a viral vector.

The invention further provides the embodiment of such composition wherein the viral vector is an adeno-associated vector, a lentiviral vector, or an adenoviral vector.

The invention further provides the embodiment of such composition wherein the composition is a pharmaceutical composition and the polynucleotide molecule encodes a vaccine to a pathogenic bacteria, virus or parasite.

The invention further provides the embodiment of such composition wherein the pathogenic bacteria, virus or parasite is selected from one or more of the group consisting of: M. pneumoniae, S. aureus, influenza, SARS-CoV-2, rubella virus, varicella zoster virus, herpes simplex, and herpes zoster.

The invention further provides the embodiment of such composition wherein the composition is a pharmaceutical composition and the polynucleotide molecule encodes a protein that is therapeutic for the treatment of a genetic disease.

The invention further provides the embodiment of such composition wherein the genetic disease is selected from the group consisting of: achondroplasia, Alzheimer's disease, alpha-1 antitrypsin deficiency, alpha-1 antitrypsin deficiency; antiphospholipid syndrome; attention deficit hyperactivity disorder (ADHD); autism; autosomal dominant polycystic kidney disease; cancer; Charcot-Marie-Tooth Disease; cri du chat syndrome; Crohn's disease, cystic fibrosis, Down syndrome, Duchenne muscular dystrophy; Factor V Leiden and Leiden thrombophilia, familial hypercholesterolemia; fragile X syndrome, Gaucher disease, Hemochromatosis; Hemophilia; Holoprosencephaly; Huntington's disease; multiple sclerosis, Parkinson's disease, phenylketonuria; severe combined immunodeficiency; sickle cell disease; spinal muscular atrophy; Tay-Sachs disease; and thalassemia.

The invention further provides a method of treating a disease caused by a pathogenic bacteria, virus or parasite, which comprises administering the composition of claim 18 to a person in need thereof, wherein the polynucleotide molecule of the composition encodes a vaccine to the pathogenic bacteria, virus or parasite.

The invention further provides the embodiment of such method wherein the pathogenic bacteria, virus or parasite is selected from one or more of the group consisting of: M. pneumoniae, S. aureus, influenza, SARS-CoV-2, rubella virus, varicella zoster virus, herpes simplex, and herpes zoster.

The invention further provides a method of treating a genetic disease which comprises administering the above-described composition to a person in need thereof wherein the polynucleotide molecule of the composition encodes a protein that is therapeutic for the treatment of the genetic disease.

The invention further provides the embodiment of such method of treating, wherein the genetic disease is selected from the group consisting of: achondroplasia, Alzheimer's disease, alpha-1 antitrypsin deficiency, alpha-1 antitrypsin deficiency; antiphospholipid syndrome; attention deficit hyperactivity disorder (ADHD); autism; autosomal dominant polycystic kidney disease; cancer; Charcot-Marie-Tooth Disease; cri du chat syndrome; Crohn's disease, cystic fibrosis, Down syndrome, Duchenne muscular dystrophy; Factor V Leiden and Leiden thrombophilia, familial hypercholesterolemia; fragile X syndrome, Gaucher disease, Hemochromatosis; Hemophilia; Holoprosencephaly; Huntington's disease; multiple sclerosis, Parkinson's disease, phenylketonuria; severe combined immunodeficiency; sickle cell disease; spinal muscular atrophy; Tay-Sachs disease; and thalassemia.

### BRIEF DESCRIPTION OF THE DRAWINGS

FIG. 1 is a map of the structural features of the illustrative rAAV vector pHelper (also known as pHelper-Kan; U.S. Pat. No. 10,557,149) that was used to illustrate the improved efficiency of transfection achieved by the present invention. The plasmid is 11,569 nucleotides in length, and provides the viral transcription and translation factors (E1a, E1b, E2a, VA and E4) required for AAV proliferation.

FIG. 2 is a map of the structural features of the illustrative rAAV vector RC Vector (also known as RC2; U.S. Pat. No. 10,557,149) that was used to illustrate the improved efficiency of transfection achieved by the present invention. The plasmid is 7,415 nucleotides in length, and provides the AAV2 rep and cap genes.

FIG. 3 is a map of the structural features of the illustrative rAAV vector pAAV2-CMV-GFP (also known as pAV-CMV-EGFP; U.S. Pat. No. 10,557,149) that was used to illustrate the improved efficiency of transfection achieved by the present invention. The plasmid is 5,030 nucleotides in length, and is designed to express the enhanced green fluorescent protein (EGFP).

FIGS. 4A-4C show the titers of transfected suspension HEK293 cells (FIG. 4A), suspension 293T cells (FIG. 4B), and adherent 293 T cells (FIG. 4C) found in the crude cell pellets. Titers were measured 48 hours post transfection with rAAV vector pAAV2-CMV-GFP, and either the PEG-LPEI conjugate (“pPEI”) of the present invention or a commercially available PEI transfection reagent that lacks PEG:PEIMax® (Polysciences) or PEIPro® (Polyplus) (at a 2:1 or 1.5:1 ratio of such reagent to DNA). The reported titers are averaged from three independent samples 48 hours post transfection.

### DETAILED DESCRIPTION OF THE INVENTION

The present invention is directed to improved methods for conjugating polyethylene glycol to polyethyleneimine, and to the use of such polyethylene glycol—polyethyleneimine conjugates to improve the efficiency with which viral and non-viral nucleic acid vectors transfect cells to provide gene therapy, and to improve the efficiency of producing viral particles that comprise therapeutic polynucleotides for use in gene therapy.

### I. Polyethylenimine (“PEI”)

As used herein, the term “polyethylenimine” (“PEI”) refers to an organic, cationic, polyamine polymer that may have primary, secondary, and/or tertiary amino groups. PEI may have a molar mass ranging from 1,000 g/mol to 100,000 g/mol. In the present invention, PEI may be in a linear form as represented by the Formula 1 (wherein n is an integer equal to or greater than 1):

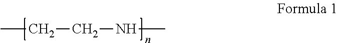

or may be a branched-type form as represented by the Formula 2 (wherein n is an integer equal to or greater than 1, and a may vary independently for each n, and is 0 or an integer equal to or greater than 1):

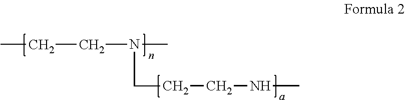

or a further branched variant thereof, in which one or more secondary amine of Formula 2 is a tertiary amine conjugated to PEI, for example, as represented by Formula 3 (wherein n is an integer equal to or greater than 1, and b and c may vary independently for each n, and is 0 or an integer equal to or greater than 1, and d may vary independently for each c, and is 0 or an integer equal to or greater than 1):

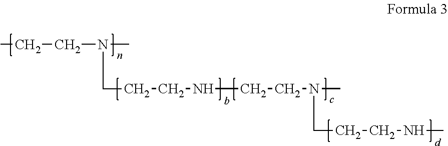

PEI has a low molecular weight, specifically ranging from 50 Da to 10,000 Da (based on the weight-average molecular weight). PEI is soluble in water, alcohol, glycol, dimethylformamide, tetrahydrofuran, esters, etc., but is insoluble in high molecular weight hydrocarbons, oleic acid, and diethyl ether.

In the context of facilitating gene transfer of non-viral vectors, the PEI-plasmid DNA (PEI-pDNA) complex is believed to enter the cell by endocytosis. PEI is believed to possess a buffering capacity and an ability to swell when protonated. Therefore, at low pH values, it is believed that PEI prevents acidification of the endosome and induces a large inflow of ions and water, subsequently leading to rupture of the endosomal or lysosomal membrane so that the PEI-plasmid DNA (PEI-pDNA) complex is delivered to the cytoplasmic space. Because lysosomal degradation is a critical hurdle for transgene expression by non-viral vectors, PEI's amelioration of such degradation is believed to cause it to have a high transfection efficiency relative to polymeric non-viral vectors such as poly-L-lysine (PLL). PEI has also been reported to undergo nuclear localization while retaining an ordered structure, once endocytosed. Because of the cationic, charge-mediated transfection, PEI introduces non-viral vector genetic materials into cells in a nonspecific manner. As for other cationic pDNA complexes, the PEI-pDNA complex transfects mainly the vascular endothelial cells of the lung after intravenous injection and this could be an obstacle to in vivo gene transfer to other types of cells.

In addition to its use as a cationic facilitator of viral and non-viral nucleic acid transfection, PEI is also used in medicinal chemistry for purposes other than gene therapy, such as an anti-inflammatory agent (Dong, L. et al. (2010) “Anti-Arthritis Activity Of Cationic Materials,” J. Cell Mol. Med. 14(7):2015-2024) or an antibacterial agent (Xu, D. et al. (2016) “Polyethyleneimine Capped Silver Nanoclusters As Efficient Antibacterial Agents,” Int. J. Environ. Res. Public Health 13(3):334:1-11; Giano, M. C. et al. (2014) “Injectable Bioadhesive Hydrogels With Innate Antibacterial Properties,” Nat. Commun. 5:4095:1-22). Medicinal chemistry uses of PEI are reviewed by Vicennati, P. et al. (2008) (“Polyethylenimine In Medicinal Chemistry,” Curr. Med. Chem. 5(27):2826-2839), and the PEG-conjugated PEI of the present invention may also be used for such purposes.

Among the various types of polymer gene carriers, branched polyethyleneimine (“BPEI”) is effective for delivering genes because the BPEI/DNA polyplexes exhibit great transgene expression both in vitro and in vivo (see, e.g., Choi, J. H. et al. (2001) “Effect of Poly(ethylene glycol) Grafting on Polyethylenimine as a Gene Transfer Vector in vitro,” Bull. Korean Chem. Soc. 22(1):46-52; Pollard, H. et al. (1998) “Polyethyleneimine But Not Cationic Lipids Promotes Transgene Delivery To The Nucleus In Mammalian Cells,” J. Biol. Chem. 273:7507-7511; Abdallah, B. et al. (1996) “A Powerful Nonviral Vector For In Vivo Gene Transfer Into The Adult Mammalian Brain: Polyethyleneimine,” Hum. Gene Ther. 7:1947-1954). Low molecular weight (600 Da) branched polyethylenimine (“600 Da BPEI”) has been used to facilitate the delivery of the β-lactam-targeting antibiotics, oxacillin and piperacillin, to wounds associated with biofilms of antimicrobial-resistant (AMR) bacteria, such as methicillin-resistant Staphylococcus aureus (MRSA), methicillin-resistant S. epidermidis (MRSE), and multi-drug resistant Pseudomonas aeruginosa (MDR-PA). It has been reported that modifying the 600 Da BPEI composition to comprise polyethylene mitigate toxicity issues associated with such compositions (Lam, A. K. et al. (2020) “PEGylation of Polyethylenimine Lowers Acute Toxicity while Retaining Anti-Biofilm and β-Lactam Potentiation Properties against Antibiotic-Resistant Pathogens,” ACS Omega. 5(40):26262-26270).

### II. Synthesis of Linear Polyethyleneimine (LPEI)

Linear PEI (“LPEI”) has a transfection activity that is as high as that of BPEI, but its cytotoxicity is lower (Jeong, J. H. et al. (2001)“DNA Transfection Using Linear Poly(Ethylenimine) Prepared By Controlled Acid Hydrolysis Of Poly(2-Ethyl-2-Oxazoline),” J. Controlled Release 73:391-399). Due to such lower cytotoxicity, linear polyethyleneimine (LPEI) is the preferred polyethyleneimine backbone polymer of the present invention.

LPEI can be obtained by the cationic ring-opening polymerization of N(2-tetrahydropyranyl)aziridine, followed by acidic hydrolysis of the corresponding substituted polyamine (US Patent Publn. US 2017/0204224 A1; Weyts, K. F. et al. (1988) “New Synthesis Of Linear Polyethyleneimine,” Polymer Bulletin 19:13-19; see also, Hsiue, G.-H. et al. (2006) “Nonviral Gene Carriers Based On Diblock Copolymers Of Poly(2-Ethyl-2-Oxazoline) And Linear Polyethylenimine,” Bioconjugate Chem. 17:781-786).

Most preferably, however, LPEI can be obtained through the hydrolysis of poly(2-ethyl-2-oxazoline) (PEOz) as shown in Reaction 1 (US Patent Publn. US 2010/0197888 A1; Tauhardt, L. et al. (2011) “Linear Polyethyleneimine: Optimized Synthesis and Characterization—On the Way to “Pharmagrade” Batches,” Macromolec. Chem. Physics 212(17):1918-1924; Fernandes, J. C. et al. (2010) “Linear Polyethylenimine Produced By Partial Acid Hydrolysis Of Poly(2-Ethyl-2-Oxazoline) For DNA And siRNA Delivery in vitro,” Gene Therapy For Inborn Errors Of Metabolism 18(Supp 1:S295; Fernandes, J. C. et al. (2013) “Linear Polyethylenimine Produced By Partial Acid Hydrolysis Of Poly(2-Ethyl Oxazoline) For DNA And siRNA Delivery in vitro,” Int. J. Nanomedicine 8:4091-4102; Ogris, M. et al. (2012) “Synthesis Of Linear Polyethylenimine And Use In Transfection,” Cold Spring Harb. Protoc. 2012(2):246-250). Accordingly, PEOz may be prepared by charging 2-ethyl-2-oxazoline, acetonitrile, and methyl p-tosylate in a round bottom flask equipped with a N₂-filled condenser and rubber stopper via an oxygen-free syringe at room temperature. After mixing, the flask is immersed in an oil bath preheated to 65° C., stirred and polymerization is allowed to continue. A 1 M KOH methanol solution is added to quench poly(2-isopropyl-2-oxazoline) oxazolinium living end groups, and the termination reaction is permitted to proceed. Thereafter, the polymerization solution is cooled to room temperature, diluted with deionized water and dialyzed against deionized water to remove oxazoline and yield the purified polymer. The PEOz is converted to LPEI via acid hydrolysis (e.g., 6 N HCL) (Fernandes, J. C. et al. (2013) “Linear Polyethylenimine Produced By Partial Acid Hydrolysis Of Poly(2-Ethyl-2-Oxazoline) For DNA And siRNA Delivery in vitro,” Int. J. Nanomedicine 8:4091-4102; Mees, M. A. et al. (2018) “Full And Partial Hydrolysis Of Poly(2-Oxazoline)s And The Subsequent Post-Polymerization Modification Of The Resulting Polyethylenimine (Co)Polymers,” Polym. Chem. 9:4968-4978):

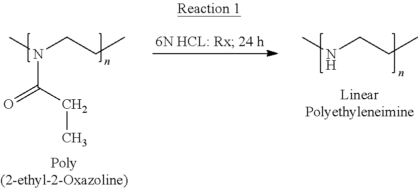

wherein n is an integer equal to or greater than 1.

The linear polyethyleneimine may then be conjugated with polyethylene glycol to form preferred conjugate (IIIa):

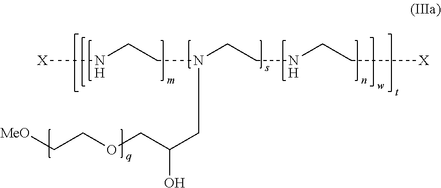

which is composed of w sets of m and n non-conjugated PEI substituents and s PEG-conjugated substituents, wherein:
R₁ is a carbon-containing, hydroxyl-comprising group that may comprise, for example, one, two, three, or more than three, carbon atoms, and that is preferably not an ethyleneimine (—CH₂—CH₂—NH—) group;
each X is independently amine, methyl or methoxy, or an alternative terminating atom or chemical group;
m and n are independently zero or an integer;
s, w and t are independently an integer equal to or greater than 1;
wherein m, n, and s may be the same, or may vary independently, for each w; and
q is an integer equal to or greater than 1, and may vary independently for each s.

### III. Conjugation of PEG with PEI

Since PEG is non-ionic and water-soluble, co-polymers of PEG and PEI exhibit improved biocompatibility, reduced cytotoxicity, and increased circulation time in vivo (Neu, M. et al. (2007) “Bioreversibly Crosslinked Polyplexes Of PEI And High Molecular Weight PEG Show Extended Circulation Times in vivo,” J. Control Release 124(1-2):69-80; Mao, S. et al. (2006) “Influence of Polyethylene Glycol Chain Length on the Physicochemical and Biological Properties of Poly(ethylene imine)-graft-Poly(ethylene glycol) Block Copolymer/SiRNA Polyplexes,” Bioconjugate Chem. 17(5):1209-1218; Zhang, X. et al. (2008) “Poly(Ethylene Glycol)-Block-Polyethylenimine Copolymers As Carriers For Gene Delivery: Effects Of PEG Molecular Weight And Pegylation Degree,” J. Biomed. Mater. Res. A. 84(3):795-804). PEG chain length and graft density of PEG was found to strongly influence siRNA condensation and polyplex stability in vitro (Neu, M. et al. (2007) “Bioreversibly Crosslinked Polyplexes Of PEI And High Molecular Weight PEG Show Extended Circulation Times in vivo,” J. Control Release 124(1-2):69-80). Preliminary in vitro investigations into the delivery of PEG-PEI polyplexes into the lungs of mice suggested that PEI/siRNA without PEG had the least immunogenicity and best stability, but these results were not supported by subsequent in vivo knockdown experiments (Mao, S. et al. (2006) “Influence of Polyethylene Glycol Chain Length on the Physicochemical and Biological Properties of Poly(ethylene imine)-graft-Poly(ethylene glycol) Block Copolymer/SiRNA Polyplexes,” Bioconjugate Chem. 17(5):1209-1218). In secondary experiments, PEG-PEI/siRNA complexes showed higher stability and elevated immune responses but no histological abnormalities, while unmodified PEI/siRNA complexes deposited PEI in the lungs and released the siRNA payload too early (Mao, S. et al. (2006) “Influence of Polyethylene Glycol Chain Length on the Physicochemical and Biological Properties of Poly(ethylene imine)-graft-Poly(ethylene glycol) Block Copolymer/SiRNA Polyplexes,” Bioconjugate Chem. 17(5):1209-1218).

Multiple different co-polymers of PEG and PEI have been described (Neu, M. et al. (2005) “Recent Advances In Rational Gene Transfer Vector Design Based On Poly(Ethylene Imine) And Its Derivatives,” J. Gene Med. 7(8):992-1009; Petersen, H. et al. (2002) “Polyethylenimine-Graft-Poly(Ethylene Glycol) Copolymers: Influence Of Copolymer Block Structure On DNA Complexation And Biological Activities As Gene Delivery System,” Bioconjug. Chem. 13(4):845-854; Banerjee, P. et al. (2006) “Linear Polyethyleneimine Grafted To A Hyperbranched Poly(Ethylene Glycol)-Like Core: A Copolymer For Gene Delivery,” Bioconjug Chem. 2006 January-February; 17(1):125-131; Singarapu, K. et al. (2013) “Polyethylene Glycol-Grafted Polyethylenimine Used To Enhance Adenovirus Gene Delivery,” J. Biomed. Mater. Res. A. 101(7):1857-1864).

For example, Petersen, H. et al. (2002) (“Synthesis, Characterization, and Biocompatibility of Polyethylenimine-graft-poly(ethylene glycol) Block Copolymers” Macromolecules 35(18):6867-6874) disclose a PEG-PEI conjugate:

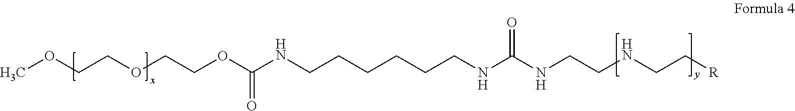

Sung, S. J. et al. (2003) (“Effect Of Polyethylene Glycol On Gene Delivery Of Polyethylenimine,” Biol. Pharm. Bull. 26(4):492-500) disclose a PEG-PEI conjugate:

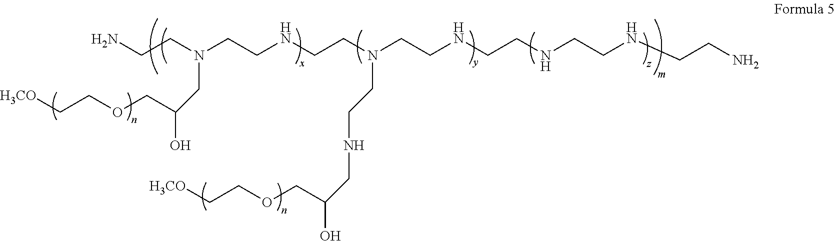

Kunath, K. et al. (2003) (“Integrin Targeting Using RGD-PEI Conjugates For In Vitro Gene Transfer,” J. Gene Med. 5(7):588-599) disclose a PEG-PEI conjugate:

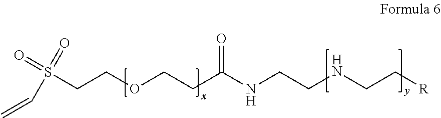

and Nguyen, H. K. et al. (2000) (“Evaluation Of Polyether-Polyethyleneimine Graft Copolymers As Gene Transfer Agents,” Gene Ther. 7(2):126-138) disclose a PEG-PEI conjugate:

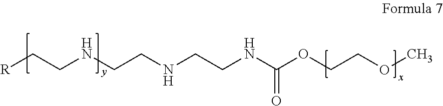

wherein x and y are independently equal to or greater than 1.

Lam, A. K. et al. (2020) (“PEGylation of Polyethylenimine Lowers Acute Toxicity while Retaining Anti-Biofilm and β-Lactam Potentiation Properties against Antibiotic-Resistant Pathogens,” ACS Omega 5(40):26262-26270) disclose a PEG-600 Da BPEI composition that is formed by reacting the epoxy group of a polyethylene glycol monoglycidyl epoxide with one of the amine groups of 600 Da BPEI”.

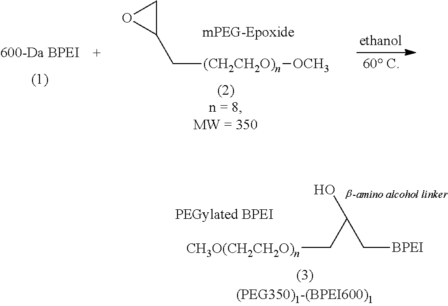

One aspect of the present invention is the development of a novel PEG-LPEI conjugate, referred to herein as “pPEI,” in which PEI, and especially linear polyethylene (LPEI), is modified to comprise PEG substituents. The general structure of such pPEI PEG-PEI conjugate is structure (III), which illustrates the preferred embodiment of pPEI in which the PEI is LPEI:

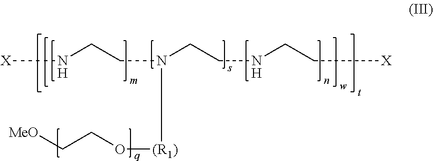

which is composed of w sets of m and n non-conjugated PEI substituents and s PEG-conjugated substituents, wherein:
R₁ is a carbon-containing, hydroxyl-comprising group that may comprise, for example, one, two, three, or more than three, carbon atoms, especially a C₁-C₃ hydroxyalkyl, and that is preferably not an ethyleneimine (—CH₂—CH₂—NH—) group;
each X is independently amine, methyl or methoxy, or an alternative terminating atom or chemical group;
m and n are independently zero or an integer;
s, w and t are independently an integer equal to or greater than 1;
wherein m, n, and s may be the same, or may vary independently, for each w; and q is an integer equal to or greater than 1, and may vary independently for each s.

The preferred pPEI PEG-PEI conjugate of the present invention is the pPEI PEG-LPEI (IIIa), wherein R₁ is —CH₂—CH(OH)—CH₂—:

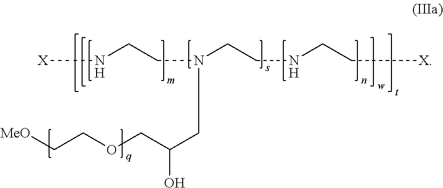

A second aspect of the present invention is the development of a novel method for producing such pPEI PEG-PEI conjugates, and particularly, pPEI PEG-LPEI (IIIa). In such method, a polyethyleneimine HCl starting material is treated with triethylamine to yield polyethyleneimine (I), comprising t ethyleneimine monomeric substituents and terminal NH₂ groups, wherein t is an integer greater than or equal to 1:

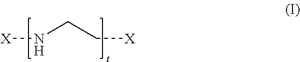

Polyethyleneimine (I) is then reacted with a polyethylene glycol derivative (II):

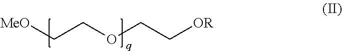

wherein q is an integer equal or greater than 1, and R is an activated leaving group that reacts with a secondary amine N of (I) to form R₁. A preferred activated leaving group R is an oxirane:

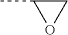

A preferred R is 2-methyloxirane is

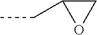

A preferred reaction, conducted in the presence of methanol and triethylamine at room temperature, or in the presence of methanol/33% isopropyl alcohol at 60° C. for 4 hours, is shown below (Reaction 2):

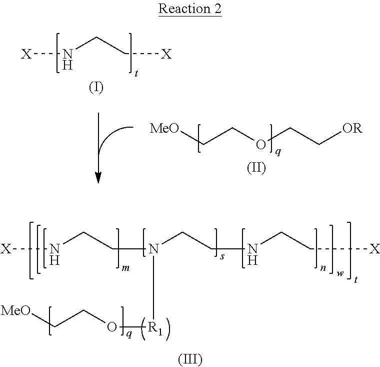

wherein t, q, m, s, n, w, q, X, and R and R₁ are as defined above.

In an alternative reaction, substituent (II) may be a C₁₂-C₁₈ carboxylic acid (e.g., lauric acid, myristic acid, palmitic acid, stearic acid, etc.):

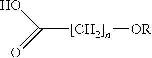

wherein n is 11-17, for example:

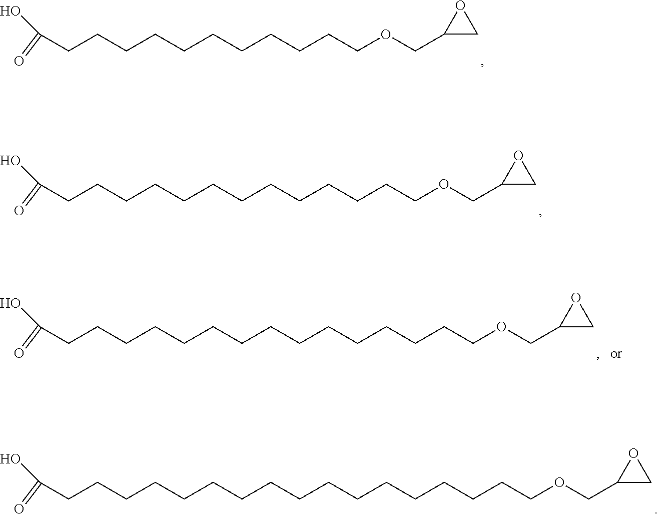 [SMILES: O=C(O)CCCCCCCCCCCOCC1CO1 ; O=C(O)CCCCCCCCCCCCCOCC1CO1 ; O=C(O)CCCCCCCCCCCCCCCOCC1CO1 ; O=C(O)CCCCCCCCCCCCCCCCCOCC1CO1]

As indicated above, a preferred activated leaving group is an oxirane, and a preferred PEG (II) is polyethylene glycol-2-(methoxy)oxirane. Polyethylene glycol-2-(methoxy)oxirane may be synthesized by reacting polyethylene glycol with 2-(chloromethyl)oxirane (also known as epichlorohydrin), to form mPEG-O-Glycidyl ether (polyethylene glycol-2-methoxyoxirane), as shown in Reaction 3:

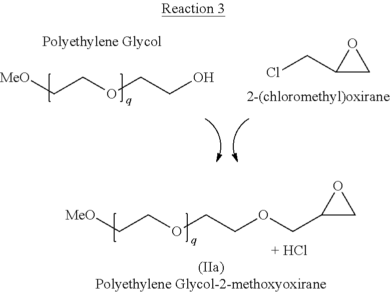

Alternatively, mPEG-O-Glycidyl ether (polyethylene glycol-2-(methoxy)oxirane) may be formed by reacting glycidol and sodium hydride with methoxypoly(ethylene glycol) methylsulfonate (“mPEG-OMS”):

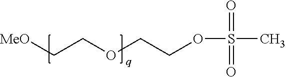

dissolved in dry tetrahydrofuran (“THF,” oxolane) at room temperature, as shown in Reaction 4:

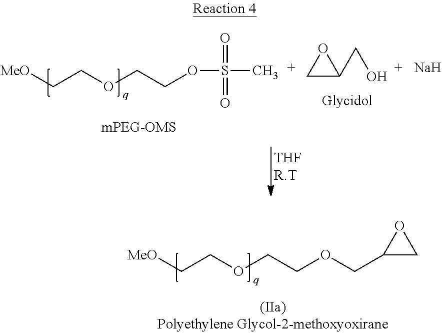

The nitrogen of a secondary amine of PEI may then react with the polyethylene glycol-2-(methoxy)oxirane to form a preferred PEG-conjugated PEI (IIIa) as shown in Reaction 5 (in which (I) and (IIIa) are incubated in methanol/33% isopropyl alcohol at 60° C. for 4 hours).

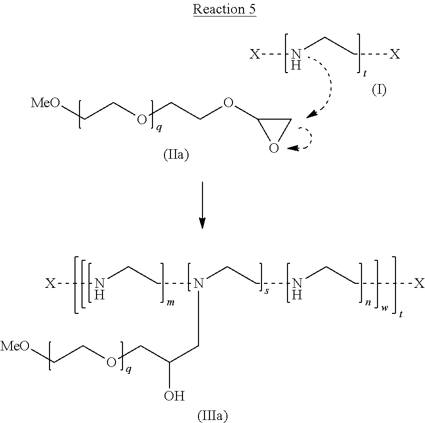

The PEI component of such conjugate will preferably have a molecular weight that ranges from a mass of 1 mer (mol. wt.=74) to a mass of approximately 2,325 mer (mol. wt.=100,000). The PEG component of such conjugate will preferably range from a mass ratio of 1 mer (mol. wt.=44) PEG to 1 mer PEI to approximately 2,272 mer PEG (mol. wt.=100,000) to 1 mer PEI. Thus, in a preferred embodiment, the conjugation of PEG to PEI may vary from approximately 1% to approximately 2,272%. In a preferred embodiment, the reaction ratio for forming such conjugates will comprise a molar ratio of 10% mPEG to LPEI (i.e., a LPEI conjugate comprising 10 monomeric substituents of PEG per 100 monomeric substituents of PEI), which provides optimized transfection efficiency and AAV and lentivirus productions, however, greater or lesser ratios of mPEG to PEI (L) may alternatively be employed.

The above-described desired PEG-conjugated LPEI is soluble in methyl alcohol (MeOH) as a free base, but becomes insoluble in MeOH upon addition of acid (e.g., HCl). As a consequence, the desired PEG-conjugated LPEI, pPEI PEG-LPEI, may be readily purified by acidifying the reaction (e.g., with HCl), and recovering the insoluble fraction.

### IV. Gene Therapy

The above-described PEG-conjugated PEI is particularly useful for increasing the efficiency of mediating gene or polynucleotide transfer relative to that observed when non-conjugated PEI of similar molecular weight and similar branching is employed. In one embodiment, such use is to enhance the efficiency with which non-viral vectors (e.g., plasmids, linear polynucleotides, etc.) transfect target cells (either in culture, ex vivo or in vivo. Without intending to be bound to any particular mechanism, the increased efficiency of mediating gene or polynucleotide transfer provided by the LPEI compounds of the present invention is believed to reflect an increase in the efficiency of gene delivery, or a decrease in the toxicity of the transfection reagents, or a combination of such, or other, mechanisms.

As used herein, the term “increased efficiency of gene delivery” is intended to denote an increase in the titer of recipient cells that have been transfected with a polynucleotide in conjunction with the PEG-conjugated PEI of the present invention, relative to the titer of recipient cells observed after transfection with such polynucleotide in conjunction with PEI of similar solubility and branching that is not conjugated to PEG. Preferably, such increased efficiency is at least 20% greater, more preferably at least 40% greater, more preferably at least 50% greater, more preferably at least 60% greater, more preferably at least 70% greater, more preferably at least 80% greater, more preferably at least 90% greater, more preferably at least 100% greater, more preferably at least 150% greater, more preferably at least 200% greater, more preferably at least 250% greater, more preferably at least 300% greater, more preferably at least 350% greater, more preferably at least 400% or more greater than the efficiency of transfection obtained using PEI lacking PEG conjugates.

As used herein, the term “decreased toxicity of transfection” is intended to denote an increase in the titer of viable recipient cells after transfection with a polynucleotide in conjunction with the PEG-conjugated PEI of the present invention, relative to the titer of viable recipient cells observed after transfection with such polynucleotide in conjunction with PEI having similar solubility and branching that is not conjugated to PEG. Preferably, such increased viability is at least 20% greater, more preferably at least 40% greater, more preferably at least 50% greater, more preferably at least 60% greater, more preferably at least 70% greater, more preferably at least 80% greater, more preferably at least 90% greater, more preferably at least 100% greater, more preferably at least 150% greater, more preferably at least 200% greater, more preferably at least 250% greater, more preferably at least 300% greater, more preferably at least 350% greater, more preferably at least 400% or more greater than the viability obtained after use of non-conjugated PEI of similar molecular weight and similar branching to the PEG-conjugated PEI of the present invention, but lacking such PEG conjugates.

In a second embodiment, such use is to enhance the efficiency of mediating gene or polynucleotide transfer of viral vectors (e.g., AAV vectors, lentiviral vectors, AD vectors, etc.) by masking viral surfaces, thereby decreasing antibody neutralization and reducing unwanted interactions with blood components after systemic administration to a recipient patient. Such masking may be accomplished by cross-linking the PEG-conjugated PEI to the surface of the viral particle via amine functional groups on the viral particle surface (for example, approximately 1,800 free amines are present on the hexon, penton and fiber proteins of Alzheimer's disease particles) (Kim, J. et al. (2012) “Enhancing The Therapeutic Efficacy Of Adenovirus In Combination With Biomaterials,” Biomaterials 33(6):1838-1850; Choi, J. W. et al. (2015) “Tuning Surface Charge and PEGylation of Biocompatible Polymers for Efficient Delivery of Nucleic Acid or Adenoviral Vector,” Bioconjug. Chem. 26(8):1818-1829; Sun, Y. et al. (2019) “Exploring The Functions Of Polymers In Adenovirus-Mediated Gene Delivery: Evading Immune Response And Redirecting Tropism,” Acta Biomater. 97:93-104; Kreppel, F. et al. (2008) “Modification Of Adenovirus Gene Transfer Vectors With Synthetic Polymers: A Scientific Review And Technical Guide,” Mol. Ther. 16(1):16-29).

As used herein, “gene therapy” is the introduction, removal, or change in the content of a recipient's genome (e.g., the genome of a human, bovine, canine, feline, equine, porcine, simian, or other mammalian recipient) via the provision of one or more polynucleotide molecules to accomplish gene addition, gene supplementation, gene editing and/or gene silencing with the goal of treating or curing a disease caused by a pathogen, or a genetic disease.

The polynucleotide molecules that may be provided to recipients in accordance with the present invention include deoxyribonucleotides or ribonucleotides and mixtures and polymers thereof in single- or double-stranded form. The term encompasses nucleic acids containing known nucleotide analogs or modified backbone residues or linkages, which are synthetic, naturally-occurring, and non-naturally-occurring, which have similar binding properties as a reference nucleic acid, and which are metabolized in a manner similar to a reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The nucleic acid may be in the form of an antisense molecule, for example a “gap-mer” containing an RNA-DNA-RNA structure that activates RNAse H. The nucleic acid can be, for example, DNA or RNA, or an RNA-DNA hybrid, and can be an oligonucleotide, plasmid, a part of a plasmid DNA, a pre-condensed DNA, a product of a nucleic acid amplification reaction, a viral vector, an expression cassette, a chimeric sequence, chromosomal DNA, or a derivative of such groups, or other form of nucleic acid molecule. The polynucleotide molecules may be a double-stranded RNA molecule of the type used for inhibiting gene expression by RNA interference. The nucleic acid may be a short interfering double-stranded RNA molecule (siRNA). The nucleic acid molecule can also be a Stealth™ RNAi molecule (Invitrogen Corporation/Life Technologies Corporation, Carlsbad, Calif.). The invention particularly contemplates that the provided polynucleotides will be an adeno-associated virus (AAV) plasmid vector, a lentiviral (e.g., HIV-1) plasmid vector, or an adenoviral (AD) plasmid vector.

The term “gene addition” refers to the provision of one or more polynucleotide(s) that had not been previously present in such recipient, e.g., polynucleotides that comprise a vaccine (e.g., a vaccine to a pathogenic bacteria, virus or parasite, or that encode a protein (such as a hormone, growth factor, etc. that is not being expressed in the recipient)). Examples of diseases caused by a pathogen include diseases caused by pathogenic bacteria (B. anthracis, Borrelia sp., C. difficile, L. pneumophila, M. pneumoniae, N. gonorrhoeae, S. aureus, T. pallidum, V. cholerae, Y. pestis, etc.), pathogenic viruses (e.g., influenza, SARS-CoV-2, rubella virus, varicella zoster virus, herpes simplex, herpes zoster) and pathogenic fungi and protozoa (e.g., Plasmodium parasites, Schistosoma parasites, Wuchereria bancrofti, Ascaris lumbricoides, etc.). Examples of genetic diseases include achondroplasia, Alzheimer's disease, alpha-1 antitrypsin deficiency, alpha-1 antitrypsin deficiency; antiphospholipid syndrome; attention deficit hyperactivity disorder (ADHD); autism; autosomal dominant polycystic kidney disease; cancer (e.g., breast cancer, colon cancer, lung cancer, prostate cancer, skin cancer, etc.); Charcot-Marie-Tooth Disease; cri du chat syndrome; Crohn's disease, cystic fibrosis, Down syndrome, Duchenne muscular dystrophy; Factor V Leiden and Leiden thrombophilia, familial hypercholesterolemia; fragile X syndrome, Gaucher disease, Hemochromatosis; Hemophilia; Holoprosencephaly; Huntington's disease; multiple sclerosis, Parkinson's disease, phenylketonuria; severe combined immunodeficiency; sickle cell disease; spinal muscular atrophy; Tay-Sachs disease; thalassemia, etc.), ameliorating the present or anticipated symptoms of such disease, or preventing the onset of such disease (see, Maestro, S. et al. (2021) “Novel Vectors And Approaches For Gene Therapy In Liver Diseases,” JHEP Rep. 3(4):100300:1-14; Nakagami, H. et al. (2021) “Therapeutic Vaccine For Chronic Diseases After The COVID-19 Era,” Hypertens. Res. 8:1-7; Pawlowski, C. et al. (2021) “FDA-Authorized mRNA COVID-19 Vaccines Are Effective Per Real-World Evidence Synthesized Across A Multi-State Health System,” Med. (NY) 2:1-14: 1-24; Tse, L. V. et al. (2020) “The Current and Future State of Vaccines, Antivirals and Gene Therapies Against Emerging Coronaviruses,” Front. Microbiol. 11:658:1-26; Colon-Thillet, R. et al. (2021) “Optimization Of AAV Vectors To Target Persistent Viral Reservoirs,” Virol. J. 18(1):85:1-18; Demminger, D. E. et al. (2020) “Adeno-Associated Virus-Vectored Influenza Vaccine Elicits Neutralizing And Fcγ Receptor-Activating Antibodies,” EMBO Mol. Med. 12(5):e10938:1-18; Rghei, A. D. et al. (2020) “AAV-Vectored Immunoprophylaxis for Filovirus Infections,” Trop. Med. Infect. Dis. 5(4):169:1-25; Tan, Z. et al. (2021) “Eliminating Mesothelioma By AAV-Vectored, PD1-Based Vaccination In The Tumor Microenvironment,” Mol. Ther. Oncolytics 20:373-386; Hasanpourghadi, M. et al. (2021) “COVID-19 Vaccines Based on Adenovirus Vectors,” Trends Biochem. Sci. 46(5):429-430; He, X. et al. (2021) “Low-Dose Ad26.COV2.S Protection Against SARS-Cov-2 Challenge In Rhesus Macaques,” Cell. 184(13):3467-3473; van der Gracht, E. T. et al. (2020) “Adenoviral Vaccines Promote Protective Tissue-Resident Memory T Cell Populations Against Cancer,” J. Immunother. Cancer. 8(2):e001133:1-12; Wang, M. et al. (2021) “Construction And Immunological Evaluation Of An Adenoviral Vector-Based Vaccine Candidate For Lassa Fever,” Viruses 13(3):484:1-15; Ferrara, F. et al. (2021) “Development of Lentiviral Vectors Pseudotyped With Influenza B Hemagglutinins: Application in Vaccine Immunogenicity, mAb Potency, and Sero-Surveillance Studies,” Front. Immunol. 12:661379:1-15; Ku, M. W. et al. (2020) “A Single Dose of NILV-Based Vaccine Provides Rapid and Durable Protection against Zika Virus,” Mol. Ther. 28(8):1772-1782; Somaiah, N. et al. (2020) “A Phase 1b Study Evaluating the Safety, Tolerability, and Immunogenicity of CMB305, a Lentiviral-Based Prime-Boost Vaccine Regimen, in Patients with Locally Advanced, Relapsed, or Metastatic Cancer Expressing NY-ESO-1,” Oncoimmunology 9(1):1847846:1-12; Toon, K. et al. (2021) “More Than Just Gene Therapy Vectors: Lentiviral Vector Pseudotypes for Serological Investigation,” Viruses 13(2):217:1-18).

In contrast, the term “gene supplementation” refers to the provision of one or more additional copies of one or more polynucleotide(s) that had been previously present in such recipient at an insufficient or undesired level. The term “gene editing” refers to the provision of one or more polynucleotide(s), wherein such provision modifies, repairs or introduces one or more polynucleotide(s) (i.e., DNA or RNA) into the recipient's cells or genome. The term “gene editing” includes the provision of polynucleotides to cause the silencing of expressed genes (e.g., polynucleotides that cause RNA interference, or that express targeted nuclease(s) (such as a zinc finger nuclease (ZFN), a transcription activator-like effector nuclease (TALEN) or may comprise a clustered regularly interspaced short palindromic repeat (CRISPR)/CRISPR associated protein 9 (CRISPR/Cas9, etc.)).

The gene therapy that may be provided in accordance with the present invention includes both ex vivo gene therapy (in which cells of the recipient are removed from a patient, provided with such polynucleotide(s)s, and then re-introduced (directly, or by their progeny after culturing and amplification) back into the recipient) and in vivo gene therapy (in which such polynucleotides are provided to the recipient (especially within a viral vector particle, liposome, etc.). Preferred viral vector particles include adeno-associated virus (AAV) vectors, HD-adenovirus vectors, or lentivirus vectors

### A. Adeno-Associated Virus (AAV) Vectors

Adeno-Associated Virus (AAV) vectors are particularly suitable for gene therapy involving polynucleotides of 4.7 kb or less. Adeno-Associated Virus (AAV) is a small (approximately 4,700 nucleotides), naturally-occurring, non-pathogenic single-stranded DNA virus belonging to the Dependovirus genus of the Parvoviridae virus (Balakrishnan, B. et al. (2014) “Basic Biology of Adeno-Associated Virus (AAV) Vectors Used in Gene Therapy,” Curr. Gene Ther. 14(2):86-100; Zinn, E. et al. (2014) “Adeno-Associated Virus: Fit To Serve,” Curr. Opin. Virol. 0:90-97). The viral genome may be described as having a 5′ portion and a 3′ portion which together comprise the genes that encode the virus' proteins (see, e.g., U.S. Pat. Nos. 10,557,149; 10,653,731; 10,801,042; 11,001,859; PCT Publns. WO 2021/011029 A1; WO 2012/011034 A1; WO 2021/011941 A1, herein incorporated by reference in their entirety).

In light of AAV's properties, recombinantly-modified versions of AAV (rAAV) have found substantial utility as vectors for gene therapy (see, Naso, M. F. et al. (2017) “Adeno-Associated Virus (AAV) as a Vector for Gene Therapy,” BioDrugs 31:317-334; Berns, K. I. et al. (2017) “AAV: An Overview of Unanswered Questions,” Human Gene Ther. 28(4):308-313; Berry, G. E. et al. (2016) “Cellular Transduction Mechanisms Of Adeno-Associated Viral Vectors,” Curr. Opin. Virol. 21:54-60; Blessing, D. et al. (2016) “Adeno-Associated Virus And Lentivirus Vectors: A Refined Toolkit For The Central Nervous System,” 21:61-66; Santiago-Ortiz, J. L. (2016) “Adeno-Associated Virus (AAV) Vectors in Cancer Gene Therapy,” J. Control Release 240:287-301; Salganik, M. et al. (2015) “Adeno-Associated Virus As A Mammalian DNA Vector,” Microbiol. Spectr. 3(4):1-32; Hocquemiller, M. et al. (2016) “Adeno-Associated Virus-Based Gene Therapy for CNS Diseases,” Hum. Gene Ther. 27(7):478-496; Lykken, E. A. et al. (2018) “Recent Progress And Considerations For AAV Gene Therapies Targeting The Central Nervous System,” J. Neurodevelop. Dis. 10:16:1-10; Buning, H. et al. (2019) “Capsid Modifications for Targeting and Improving the Efficacy of AAV Vectors,” Mol. Ther. Meth. Clin. Devel. 12:P248-P265; During, M. J. et al. (1998) “In Vivo Expression Of Therapeutic Human Genes For Dopamine Production In The Caudates Of MPTP-Treated Monkeys Using An AAV Vector,” Gene Ther. 5:820-827; Grieger, J. C. et al. (2012) “Adeno-Associated Virus Vectorology, Manufacturing, and Clinical Applications,” Meth. Enzymol. 507:229-254; Kotterman, M. A. et al. (2014) “Engineering Adeno-Associated Viruses For Clinical Gene Therapy,” Nat. Rev. Genet. 15(7):445-451; Kwon, I. et al. (2007) “Designer Gene Delivery Vectors: Molecular Engineering and Evolution of Adeno-Associated Viral Vectors for Enhanced Gene Transfer,” Pharm. Res. 25(3):489-499).

rAAV are typically produced using circular plasmids (“rAAV plasmid vector”). The AAV rep and cap genes are typically deleted from such constructs and replaced with a promoter, a β-globin intron, a cloning site into which a therapeutic gene of choice (transgene) has been inserted, and a poly-adenylation (“polyA”) site. The inverted terminal repeated sequences (ITR) of the rAAV are, however, retained, so that the transgene expression cassette of the rAAV plasmid vector is flanked by AAV ITR sequences (Colella, P. et al. (2018) “Emerging Issues in AAV-Mediated In Vivo Gene Therapy,” Molec. Ther. Meth. Clin. Develop. 8:87-104; Buning, H. et al. (2019) “Capsid Modifications for Targeting and Improving the Efficacy of AAV Vectors,” Mol. Ther. Meth. Clin. Devel. 12:P248-P265). Thus, in the 5′ to 3′ direction, the rAAV comprises a 5′ ITR, the transgene expression cassette of the rAAV, and a 3′ ITR.

Since rAAV are defective viruses, additional functions must be provided in order to replicate and package rAAV. Thus, second plasmid vector is provided, that comprises an AAV helper function-providing polynucleotide that provides the Rep52 and Rep78 genes that are required for vector transcription control and replication, and for the packaging of viral genomes into the viral capsule (Rep40 and Rep68 are not required for rAAV production) and the cap genes that were excised from the AAV in order to produce the rAAV. The second plasmid vector may additionally comprise a non-AAV helper function-providing polynucleotide that encodes the viral transcription and translation factors (E1a, E1b, E2a, VA and E4) required for AAV proliferation, so as to comprise, in concert with the rAAV, a double plasmid transfection system (Grimm, D. et al. (1998) “Novel Tools For Production And Purification Of Recombinant Adeno-Associated Virus Vectors,” Hum. Gene Ther. 9:2745-2760; Penaud-Budloo, M. et al. (2018) “Pharmacology of Recombinant Adeno-associated Virus Production,” Molec. Ther. Meth. Clin. Develop. 8:166-180).

However, it has become increasingly common to clone the AAV helper function-providing polynucleotide (which provides the required rep and cap genes) into an AAV helper plasmid, and to clone the non-AAV helper function-providing polynucleotide (which provides the genes that encode the viral transcription and translation factors) on a different plasmid (e.g., an “Ad helper plasmid”), so that such plasmids, in concert with an rAAV plasmid vector, comprise a triple plasmid transfection system. Use of the triple plasmid transfection system has the advantage of permitting one to easily switch one cap gene for another, thereby facilitating changes in the rAAV's serotype. The use of helper plasmids, rather than helper viruses, permits rAAV to be produced without additionally producing particles of the helper virus (Francois, A. et al. (2018) “Accurate Titration of Infectious AAV Particles Requires Measurement of Biologically Active Vector Genomes and Suitable Controls,” Molec. Ther. Meth. Clin. Develop. 10:223-236; Matsushita, T. et al. (1998) “Adeno-Associated Virus Vectors Can Be Efficiently Produced Without Helper Virus,” Gene Ther. 5:938-945).

The transient transfection of plasmid DNAs comprising the rAAV plasmid vector, the AAV rep and cap genes, and the trans-acting AAD helper genes into HEK293 cells by calcium phosphate coprecipitation has become the standard method to produce rAAV in the research laboratory (Grimm, D. et al. (1998) “Novel Tools For Production And Purification Of Recombinant Adeno-Associated Virus Vectors,” Hum. Gene Ther. 9:2745-2760). However, the use of such a calcium phosphate-mediated transfection process with suspension-cultured transfected mammalian cells requires media exchanges, and is thus not considered ideal for the large-scale rAAV production that is required in order to produce therapeutic doses of rAAV (Lock, M. et al. (2010) “Rapid, Simple, and Versatile Manufacturing of Recombinant Adeno-Associated Viral Vectors at Scale,” Hum. Gene Ther. 21:1259-1271). For this reason, polyethylenimine (PEI), has been used as a transfection reagent and has been found to provide yields of virus that are similar to those obtained using calcium phosphate-mediated transfection (Durocher, Y. et al. (2007) “Scalable Serum-Free Production Of Recombinant Adeno-Associated Virus Type 2 By Transfection Of 293 Suspension Cells,” J. Virol. Meth. 144:32-40).

rAAV containing a desired transgene expression cassette are typically produced by human cells (such as HEK293) grown in suspension. rAAV may alternatively be produced in insect cells (e.g., sf9 cells) using baculoviral vectors (see, e.g., U.S. Pat. No.: 9,879,282; 9,879,279; 8,945,918; 8,163,543; 7,271,002 and 6,723,551), or in HSV-infected baby hamster kidney (BHK) cells (e.g., BHK21) (Francois, A. et al. (2018) “Accurate Titration of Infectious AAV Particles Requires Measurement of Biologically Active Vector Genomes and Suitable Controls,” Molec. Ther. Meth. Clin. Develop. 10:223-236). Methods of rAAV production are reviewed in Grieger, J. C. et al. (2012) “Adeno-Associated Virus Vectorology, Manufacturing, and Clinical Applications,” Meth. Enzymol. 507:229-254, and in Penaud-Budloo, M. et al. (2018) “Pharmacology of Recombinant Adeno-associated Virus Production,” Molec. Ther. Meth. Clin. Develop. 8:166-180.

AAV vectors that may be transfected using the novel compositions and methods of the present invention for use as vaccines and in gene therapy are known in the art (see. e.g., U.S. Pat. Nos. 10,557,149; 10,653,731; 10,801,042; 11,001,859; PCT Publns. WO 2021/011029 A1; WO 2012/011034 A1; WO 2021/011941 A1; see also, Colon-Thillet, R. et al. (2021) “Optimization Of AAV Vectors To Target Persistent Viral Reservoirs,” Virol. J. 18(1):85:1-18; Demminger, D. E. et al. (2020) “Adeno-Associated Virus-Vectored Influenza Vaccine Elicits Neutralizing And Fcγ Receptor-Activating Antibodies,” EMBO Mol. Med. 12(5):e10938:1-18; Rghei, A. D. et al. (2020) “AAV-Vectored Immunoprophylaxis for Filovirus Infections,” Trop. Med. Infect. Dis. 5(4):169:1-25; Tan, Z. et al. (2021) “Eliminating Mesothelioma By AAV-Vectored, PD1-Based Vaccination In The Tumor Microenvironment,” Mol. Ther. Oncolytics 20:373-386) or may be developed in light of the above description.

### B. Lentiviral Vectors

Lentivirus vectors are particularly suitable for gene therapy involving polynucleotides of 10 kb or less. Lentiviruses are members of the retroviridae family of viruses. They include primate and non-primate retroviruses (such as HIV and SIV (simian immunodeficiency virus), FIV (feline immunodeficiency virus), BIV (bovine immunodeficiency virus), CAEV (caprine arthritis-encephalitis virus), EIAV (equine infectious anemia virus) and visnavirus) (Escors, D. et al. (2011) “Lentiviral Vectors In Gene Therapy: Their Current Status And Future Potential,” Arch. Immunol. Ther. Exp. (Warsz.) 58(2):107-119). The most widely studied lentivirus is HIV-1, the causative agent of AIDS (Danforth, K. et al. (2017) “Global Mortality and Morbidity of HIV/AIDS,” In: MAJOR INFECTIOUS DISEASES, Vol. 6 (Holmes, K. K. et al. (Eds.)) The World Bank, Washington, D.C.; Engelman, A. et al. (2012) “The Structural Biology Of HIV-1: Mechanistic And Therapeutic Insights,” Nat. Rev. Microbiol. 10(4):279-290; Freed, E. O. (2015) “HIV-1 Assembly, Release And Maturation,” Nat. Rev. Microbiol. 13(8):484-496; Soliman, M. et al. (2017) “Mechanisms of HIV-1 Control,” Curr. HIV/AIDS Rep. 14(3):101-109; Elsinger, R. W. et al. (2018) “Ending the HIV/AIDS Pandemic,” Emerg. Infect. dis. 24(3):413-416; US Patent Publn. US 2019/0211360 A1).

The wildtype lentiviral genome consists of two linear, single-stranded, positive-sense RNA molecules of 9.75 kb, whose ends are flanked by long terminal repeated sequences (LTR). These 5′ and 3′ LTR sequences are required for viral transcription, reverse transcription, and integration of the viral genome. The lentiviral genome comprises at least nine genes: gag, pol, env, tat, rev, vpu, vpr, vif and nef (Hope, T. J. et al. (2000) “Structure, Expression, and Regulation of the HIV Genome,” HIV InSite Knowledge Base Chapter, pages 1-12).

Lentiviruses cannot be directly employed in gene therapy because their capacity to integrate into cellular chromosomes of infected cells is potentially oncogenic. Thus, lentiviral vector systems have been developed that do not permit chromosomal integration to occur. Most recombinant lentivirus vectors are derived from HIV-1. In order to comport with the constraints that certain lentiviral elements, such as the LTRs, Ψ, and RRE (Rev response element required for processing and transport of viral RNAs) are required in cis, whereas other lentiviral elements, such as genes: gag, pol, env, tat, and rev function in trans, such vector systems entail the co-transfection of multiple plasmids, for example:
(1) a packaging plasmid that encoded the required gag and pol sequences, and the viral regulatory genes tat and rev;
(2) an envelope vector that encoded a non-lentiviral, heterogenous envelope protein (“pseudotyped” envelope protein) that determines the host range (tropism) of the lentiviral particle; and
(3) a transfer vector that comprised the 5′ LTR and a portion of the 3′ LTR sequence that lacks the 3′ LTR U3 region (Pincha, M. et al. (2010) “Lentiviral Vectors For Immunization: An Inflammatory Field,” Expert Rev. Vaccines 9(3):309-321). The deletion of the 3′ LTR U3 region results in the Transcriptional Self-Inactivation (SIN) of potentially packageable viral genomes in transduced cells (Schambach, A. et al. (2013) “Biosafety Features of Lentiviral Vectors,” Human Gene Ther. 24:132-142; Yu, S.F. et al. (1986) “Self-Inactivating Retroviral Vectors Designed For Transfer Of Whole Genes Into Mammalian Cells,” Proc. Natl. Acad. Sci. (U.S.A.) 83(10):3194-3198; Miyoshi, H. (1998) “Development Of A Self-Inactivating Lentivirus Vector,” J. Virol. 72(10):8150-8157). The transfer vector additionally comprises Ψ, the RRE, the central polypurine tract (cPPT) (derived from the Pol reading frame) that increases the efficiency of reverse transcription (RT), and a desired exogenous gene for expression (transgene) under the control of a promoter, such as the cytomegalovirus (CMV) immediate-early enhancer and promoter (Barrow, K. M. (2006) “Use Of The Cytomegalovirus Promoter For Transient And Stable Transgene Expression In Mouse Embryonic Stem Cells,” Methods Mol Biol. 329:283-294) or the Rous sarcoma virus (RSV) (Yaguchi, M. et al. (2013) “Characterization Of The Properties Of Seven Promoters In The Motor Cortex Of Rats And Monkeys After Lentiviral Vector-Mediated Gene Transfer,” Hum. Gene Ther. Methods 24(6):333-344).
   Co-transfection with all three vectors thus results in the production of lentiviral particles that express the pseudo-envelope protein on their surfaces. By replacing the native lentiviral envelope protein with a heterogenous “pseudotyped” envelope protein, such vector systems alter the tropism of the lentivirus so that it targets desired host cells. For example, the native lentiviral envelope protein can be replaced with the vesicular stomatitis virus glycoprotein G (VSV-G) to create a pseudotyped lentiviral vectors having extensive host cell infectivity (see, Gruber, A. et al. (2000) “Dendritic Cells Transduced By Multiply Deleted HIV-1 Vectors Exhibit Normal Phenotypes And Functions And Elicit An HIV-Specific Cytotoxic T-Lymphocyte Response in vitro,” Blood 96:1327-1333; Zufferey, R. et al. (1997) “Multiply Attenuated Lentiviral Vector Achieves Efficient Gene Delivery in vivo,” Nat. Biotechnol. 15:871-875).

Lentiviruses have thus evolved into highly efficient vehicles for in vivo gene delivery (Chen, S.-H. et al. (2019) “Overview: Recombinant Viral Vectors as Neuroscience Tools,” Curr. Protoc. Neurosci. 87(1):e67:1-16; Lundstrom, K. (2019) “RNA Viruses as Tools in Gene Therapy and Vaccine Development.” Genes (Basel) 10(3):1-24; Keeler, A. M. et al. (2017) “Gene Therapy 2017: Progress and Future Directions,” Clin. Transl. Sci. 10:242-248; Milone, M. C. et al. (2018) “Clinical Use of Lentiviral Vectors,” Leukemia 32:1529-1541; Escors, D. et al. (2011) “Lentiviral Vectors In Gene Therapy: Their Current Status And Future Potential,” Arch. Immunol. Ther. Exp. (Warsz.) 58(2):107-119; Schambach, A. et al. (2013) “Biosafety Features of Lentiviral Vectors,” Human Gene Ther. 24:132-142; Shirley, J. L. et al. (2020) “Immune Responses to Viral Gene Therapy Vectors,” Molec. Ther. 28(3):709-722).

Lentiviral vectors that may be transfected using the novel compositions and methods of the present invention for use as vaccines and in gene therapy are known in the art (see. e.g., PCT Application PCT/US2021/030814 and U.S. patent application Ser. No. 16/877,839, Ferrara, F. et al. (2021) “Development of Lentiviral Vectors Pseudotyped With Influenza B Hemagglutinins: Application in Vaccine Immunogenicity, mAb Potency, and Sero-Surveillance Studies,” Front. Immunol. 12:661379:1-15; Ku, M. W. et al. (2020) “A Single Dose of NILV-Based Vaccine Provides Rapid and Durable Protection against Zika Virus,” Mol. Ther. 28(8):1772-1782; Somaiah, N. et al. (2020) “A Phase 1b Study Evaluating the Safety, Tolerability, and Immunogenicity of CMB305, a Lentiviral-Based Prime-Boost Vaccine Regimen, in Patients with Locally Advanced, Relapsed, or Metastatic Cancer Expressing NY-ESO-1,” Oncoimmunology 9(1):1847846:1-12; Toon, K. et al. (2021) “More Than Just Gene Therapy Vectors: Lentiviral Vector Pseudotypes for Serological Investigation,” Viruses 13(2):217:1-18), or may be developed in light of the above description.

### C. HD-Adenoviral Vectors

Adenoviruses (Ad) have a non-enveloped icosahedral capsid of approximately 100 nm containing a linear double-stranded DNA genome of approximately 36 kb (Shenk, T. et al. (1996) In: ADENOVIRIDAE: THE VIRUSES AND THEIR REPLICATION. Fields, B. N. et al. (eds.), Lipponcott-Raven Publishers (Philadelphia); pp. 2265-2326).

The most extensively characterized adenoviruses are serotypes 2 (Ad2) and 5 (Ad5) of subgroup C. Their genome comprises cis-acting inverted terminal repeats (ITRs) which are required for viral DNA replication. A cis-acting packaging signal (Ω), required for the encapsidation of the Ad genome is located near the left ITR. The Ad genome comprises early region genes: E1A, E1B, E2, E3 and E4, which are expressed before DNA replication has occurred, and late region genes: L1-L5, which are expressed to high levels after initiation of DNA replication (Rosewell, A. et al. (2011) “Helper-Dependent Adenoviral Vectors,” J. Genet. Syndr. Gene Ther. Suppl 5:001:1-34).

Current Ad vectors are helper-dependent (HD-Ad) vectors (also referred to as high-capacity (HC-Ad) vectors) that are devoid of all viral genes except those cis-acting elements needed for vector genome replication (ITRs) and encapsidation (w). Such vectors have a transgene capacity of 36 kb, but require a helper virus that must be able to replicate normally and express all of the viral proteins needed to replicate and package the HD-Ad genome.

The most efficient method for producing HD-Ad is the Cre/loxP system (Parks, R. J. (1996) “A Helper-Dependent Adenovirus Vector System: Removal Of Helper Virus By Cre-Mediated Excision Of The Viral Packaging Signal,” Proc. Natl. Acad. Sci. (U.S.A.) 93(24):13565-13570). In this system, the HD-Ad genome, constructed in a bacterial plasmid, contains the adenoviral ITRs, which are required for vector genome replication, and w, which is the packaging signal required for encapsidation of the vector genome into the capsid. The HD-Ad plasmid additionally comprises the expression cassette of interest and additional non-coding eukaryotic “stuffer” DNA, if necessary to bring the vector genome size within the size range (27.7 kb to 37 kb) for efficient packaging into virions. To convert the HD-Ad plasmid into a viral form, the plasmid is co-transfected with the linearized HD-Ad genome into cells (e.g. HEK293 cells) expressing the site-specific Cre recombinase, and subsequently infected with an E1-deleted Ad helper virus bearing a packaging signal flanked by loxP sites. As a consequence of such infection, the packaging signal is excised from the helper virus genome by Cre-mediated site-specific recombination between the loxP sites. This renders the helper virus unpackageable but still able to undergo DNA replication and thus trans-complement the replication and encapsidation of the HD-Ad genome.

HD-Ad vectors can infect many cell types including low-proliferative or quiescent cell populations and antigen-presenting cells (APC). They exhibit low immunotoxicity and promote stable long-term transgene expression in multiple tissues (Rosewell, A. et al. (2011) “Helper-Dependent Adenoviral Vectors,” J. Genet. Syndr. Gene Ther. Suppl 5:001:1-34; Piccolo, P. et al. (2014) “Challenges and Prospects for Helper-Dependent Adenoviral Vector-Mediated Gene Therapy,” Biomedicines 2(2):132-148; Brunetti-Pierri, N. et al. (2011) “Helper-Dependent Adenoviral Vectors For Liver-Directed Gene Therapy,” Hum. Mol. Genet. 20(R1):R7-13; Józkowicz, A. et al. (2005) “Helper-Dependent Adenoviral Vectors In Experimental Gene Therapy,” Acta Biochim. Pol. 52(3):589-599; Wong, C. M. et al. (2013) “The Role Of Chromatin In Adenoviral Vector Function,” Viruses 5(6):1500-1515; Farzad, L. M. et al. (2014) “Feasibility of Applying Helper-Dependent Adenoviral Vectors for Cancer Immunotherapy,” Biomedicines 2(1):110-131; Bangari, D. S. et al. (2006) “Current Strategies And Future Directions For Eluding Adenoviral Vector Immunity,” Curr. Gene Ther. 6(2):215-226; Thorrez, L. et al. (2004) “Preclinical Gene Therapy Studies For Hemophilia Using Adenoviral Vectors,” Semin. Thromb. Hemost. 30(2):173-183; Lopez-Gordo, E. et al. (2014) “Circumventing Anti-Vector Immunity: Potential Use Of Nonhuman Adenoviral Vectors,” Hum. Gene Ther. 25(4):285-300).

Adenoviral vectors that may be transfected using the novel compositions and methods of the present invention for use as vaccines and in gene therapy are known in the art (see. e.g., Hasanpourghadi, M. et al. (2021) “COVID-19 Vaccines Based on Adenovirus Vectors,” Trends Biochem. Sci. 46(5):429-430; He, X. et al. (2021) “Low-Dose Ad26.COV2.S Protection Against SARS-Cov-2 Challenge In Rhesus Macaques,” Cell. 184(13):3467-3473; van der Gracht, E. T. et al. (2020) “Adenoviral Vaccines Promote Protective Tissue-Resident Memory T Cell Populations Against Cancer,” J. Immunother. Cancer. 8(2):e001133:1-12; Wang, M. et al. (2021) “Construction And Immunological Evaluation Of An Adenoviral Vector-Based Vaccine Candidate For Lassa Fever,” Viruses 13(3):484:1-15) or may be developed in light of the above description.

### V. Embodiments of the Invention

Having now generally described the invention, the same will be more readily understood through reference to the following numbered Embodiments (“E”), which are provided by way of illustration and are not intended to be limiting of the present invention unless specified:
E1. A polyethylene glycol conjugate of polyethyleneimine comprising the Formula (III):

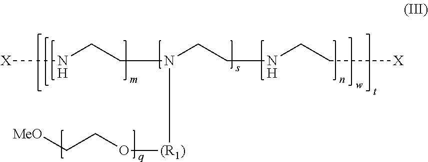

wherein: R₁ is a carbon-containing, hydroxyl-comprising group that may comprise, for example, one, two, three, or more than three, carbon atoms, and that is preferably not an ethyleneimine (—CH₂—CH₂—NH—) group;
each X is independently amine, methyl or methoxy, or an alternative terminating atom or chemical group;
m and n are independently zero or an integer;
s, w and t are independently an integer equal to or greater than 1;
wherein m, n, and s may be the same, or may vary independently, for each w; and
q is an integer equal to or greater than 1, and may vary independently for each s.
E2. The polyethylene glycol conjugate of polyethyleneimine of E1, wherein R₁ is —CH₂—CH(OH)—CH₂—, and said polyethylene glycol conjugate of polyethyleneimine (III) is:

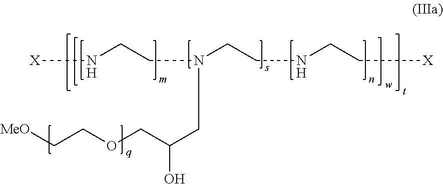

E3. The polyethylene glycol conjugate of polyethyleneimine of any one of E1-E2, wherein the polyethyleneimine of such conjugate is linear polyethyleneimine.
E4. The polyethylene glycol conjugate of polyethyleneimine of any one of E1-E2, wherein the polyethyleneimine of such conjugate is a branched polyethyleneimine.
E5. The polyethylene glycol conjugate of polyethyleneimine of any one of E1-E4, wherein said conjugate comprises from 1 to approximately 2325 ethyleneimine monomer substitutents.
E6. The polyethylene glycol conjugate of polyethyleneimine any one of E1-E5, wherein said conjugate comprises from 1 to approximately 2272 ethylene glycol substituents.
E7. The polyethylene glycol conjugate of polyethyleneimine (III) of any one of E1-E6, wherein:
   (A) from about 2% to about 100% of the ethyleneimine monomer substituents thereof are conjugated to PEG;
   (B) from about 5% to about 80% of the ethyleneimine monomer substituents thereof are conjugated to PEG;
   (C) from about 10% to about 30% of the ethyleneimine monomer substituents thereof are conjugated to PEG; or
   (D) about 10% of the ethyleneimine monomer substituents thereof are conjugated to PEG.
E8. A method of producing the polyethylene glycol conjugate of any one of E1l-E7, which comprises:
   (A) reacting polyethyleneimine of Formula (I)

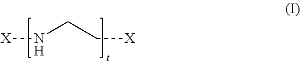

with polyethylene glycol of Formula (II)

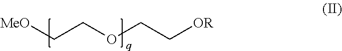

wherein R is an activated leaving group that reacts with a secondary amine N of (I) to form the carbon-containing, hydroxyl-comprising group R₁ that may comprise, for example, one, two, three, or more than three, carbon atoms;
   in MeOH at neutral or basic pH.
(B) acidifying the reaction to thereby cause said polyethylene glycol conjugate of polyethyleneimine (III) to become insoluble; and
(C) recovering said polyethylene glycol conjugate of polyethyleneimine (III) from the insoluble fraction of the reaction.
E9. The method of producing the polyethylene glycol conjugate of polyethyleneimine of

E8, wherein the polyethyleneimine of such conjugate is linear polyethyleneimine.
E10. The method of producing the polyethylene glycol conjugate of polyethyleneimine of E8, wherein the polyethyleneimine of such conjugate is a branched polyethyleneimine.
E11. The method of producing the polyethylene glycol conjugate of any one of E9-E1 wherein said polyethylene glycol of Formula (II) is polyethylene glycol-2-(methoxy)oxirane:

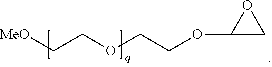

E12. A composition comprising the polyethylene glycol conjugate of polyethyleneimine (III) of any one of E1-E7, and a polynucleotide molecule.
E13. The composition of E12, wherein the polyethyleneimine of such conjugate is linear polyethyleneimine.
E14. The composition of E12, wherein the polyethyleneimine of such conjugate is a branched polyethyleneimine.
E15. The composition of any one of E12-E14 wherein said composition is suitable for transfecting said polynucleotide into a recipient cell, wherein the presence of said polyethylene glycol conjugate of polyethyleneimine (III) enhances the efficiency of transfection or the viability of transfected cells relative to the efficiency of transfection or the viability of transfected cells when transfected in the absence of said polyethylene glycol conjugate of polyethyleneimine (III).
E16. The composition of E15, wherein said efficiency of transfection or viability in the presence of said polyethylene glycol conjugate of polyethyleneimine (III) is:
   (A) at least 50% greater than the efficiency of transfection or the viability of such transfected cells when transfected in the absence of said polyethylene glycol conjugate of polyethyleneimine (III);
   (B) at least 100% greater than the efficiency of transfection or the viability of such transfected cells when transfected in the absence of said polyethylene glycol conjugate of polyethyleneimine (III);
   (C) at least 200% greater than the efficiency of transfection or the viability of such transfected cells when transfected in the absence of said polyethylene glycol conjugate of polyethyleneimine (III);
   (D) at least 300% greater than the efficiency of transfection or the viability of such transfected cells when transfected in the absence of said polyethylene glycol conjugate of polyethyleneimine (III); or
   (E) at least 400% greater than the efficiency of transfection or the viability of such transfected cells when transfected in the absence of said polyethylene glycol conjugate of polyethyleneimine (III).
E17. The composition of any one of E12-E16, wherein said polynucleotide molecule comprises a non-viral vector.
E18. The composition of E17, wherein said polynucleotide molecule comprises an RNA non-viral vector.
E19. The composition of E17, wherein said polynucleotide molecule comprises a DNA non-viral vector.
E20. The composition of E17, wherein non-viral vector is a plasmid vector.
E21. The composition of any one of E12-E16, wherein said polynucleotide molecule comprises a viral vector.
E22. The composition of E21, wherein said polynucleotide molecule comprises an RNA viral vector.
E23. The composition of E21, wherein said polynucleotide molecule comprises a DNA viral vector.
E24. The composition of E21, wherein said viral vector is an adeno-associated vector, a lentiviral vector, or an adenoviral vector.
E25. The composition of any one of E21-E24, wherein said composition is a pharmaceutical composition and said polynucleotide molecule encodes a vaccine to a pathogenic bacteria, virus or parasite.
E26. The composition of E25, wherein said pathogenic bacteria, virus or parasite is selected from one or more of the group consisting of: M. pneumoniae, S. aureus, influenza, SARS-CoV-2, rubella virus, varicella zoster virus, herpes simplex, and herpes zoster.
E27. The composition of any one of E12-E24, wherein said composition is a pharmaceutical composition and said polynucleotide molecule is therapeutic for the treatment of a genetic disease or encodes a protein that is therapeutic for the treatment of a genetic disease.
E28. The composition of E27, wherein said genetic disease is selected from the group consisting of: achondroplasia, Alzheimer's disease, alpha-1 antitrypsin deficiency, alpha-1 antitrypsin deficiency; antiphospholipid syndrome; attention deficit hyperactivity disorder (ADHD); autism; autosomal dominant polycystic kidney disease; cancer; Charcot-Marie-Tooth Disease; cri du chat syndrome; Crohn's disease, cystic fibrosis, Down syndrome, Duchenne muscular dystrophy; Factor V Leiden and Leiden thrombophilia, familial hypercholesterolemia; fragile X syndrome, Gaucher disease, Hemochromatosis; Hemophilia; Holoprosencephaly; Huntington's disease; multiple sclerosis, Parkinson's disease, phenylketonuria; severe combined immunodeficiency; sickle cell disease; spinal muscular atrophy; Tay-Sachs disease; and thalassemia.
E29. A method of treating a disease caused by a pathogenic bacteria, virus or parasite, which comprises administering the composition of E25 to a person in need thereof, wherein the polynucleotide molecule of said composition encodes a vaccine to said pathogenic bacteria, virus or parasite.
E30. The method of E29, wherein said pathogenic bacteria, virus or parasite is selected from one or more of the group consisting of: M. pneumoniae, S. aureus, influenza, SARS-CoV-2, rubella virus, varicella zoster virus, herpes simplex, and herpes zoster.
E31. A method of treating a genetic disease which comprises administering the composition of E27 to a person in need thereof wherein the polynucleotide molecule of said composition is therapeutic for the treatment of said genetic disease, or encodes a protein that is therapeutic for the treatment of said genetic disease.
E32. The method of E31, wherein said genetic disease is selected from the group consisting of: achondroplasia, Alzheimer's disease, alpha-1 antitrypsin deficiency, alpha-1 antitrypsin deficiency; antiphospholipid syndrome; attention deficit hyperactivity disorder (ADHD); autism; autosomal dominant polycystic kidney disease; cancer; Charcot-Marie-Tooth Disease; cri du chat syndrome; Crohn's disease, cystic fibrosis, Down syndrome, Duchenne muscular dystrophy; Factor V Leiden and Leiden thrombophilia, familial hypercholesterolemia; fragile X syndrome, Gaucher disease, Hemochromatosis; Hemophilia; Holoprosencephaly; Huntington's disease; multiple sclerosis, Parkinson's disease, phenylketonuria; severe combined immunodeficiency; sickle cell disease; spinal muscular atrophy; Tay-Sachs disease; and thalassemia.

### EXAMPLES

Having now generally described the invention, the same will be more readily understood through reference to the following examples, which are provided by way of illustration and are not intended to be limiting of the present invention unless specified.

### Example 1

### Conjugation PEG to PEI (pPEI) for Plasmid DNA Delivery and Virus Production

In order to demonstrate the ability of the PEG-PEI conjugates of the present invention to improve the efficiency with which viral and non-viral nucleic acid vectors transfect cells, a PEG-LPEI conjugate was prepared and used to transfect cells with three AAV packaging plasmids in order to produce rAAV particles.

To form the PEG-PEI conjugate, mPEG-O-Glycidyl ether (polyethylene glycol-2-methoxyoxirane) was prepared by dissolving glycidol (having a specific gravity of 1.18) in a 250-mL round bottom flask equipped with magnetic stirrer at 0° C. Sodium hydride (NaH) (60% oil dispersion; 0.767 g) was added pinch by pinch. After the addition of the NaH, the solution was stirred for 1 h at room temperature. Methoxypoly(ethylene glycol) methylsulfonate (“mPEG-OMS”) (7.71 g) dissolved in dry tetrahydrofuran (“THF,” oxolane) (30 mL) was then added dropwise and the reaction mixture was stirred at room temperature overnight. The reaction was considered to have reached completion via TLC analysis (100:1; CHCl₃:MeOH: 5% molibidic phosphate). The solvent was removed under reduced pressure and the crude oil was mixed with isopropyl alcohol (“IPA”) (1 ml), followed by addition of water. The aqueous layer was extracted with dichloromethane (“DCM”) (3×50 mL); washed with water (1, brine (1×200 mL); dried with MgSO₄ and concentrated under reduced pressure to yield the mPEG-O-Glycidyl ether (polyethylene glycol-2-methoxyoxirane) (6 g) as an almost pure (90%) colorless liquid (see, Reaction 4):

The mPEG-O-Glycidyl ether (polyethylene glycol-2-methoxyoxirane) was then conjugated with PEI as described above (see, Reaction 5). Specifically, 1 g of linear PEI was introduced into a round bottom flask, to which MeOH (10 mL) was added with stirring under nitrogen. The resultant compound does not dissolve. Triethylamine (TEA) (4mL) was then added, and the solution became clear. mPEG glycidyl ether (0.346 g), dissolved in a mixture of isopropyl alcohol (IPA) : water (5:1) was added to the stirring clear solution. The reaction mixture was then stirred at 70-80° C. for 3 h. The progress of the reaction was monitored by TLC (5% MeOH: chloroform) until no mPEG-glycidyl ether remained. Solvent was then removed under reduced pressure. The product was then again dissolved in dry methanol, and cooled in an ice bath with methanolic HCl. The resultant product was found to be insoluble in methanol but a TEA.HCl by-product was soluble in methanol. The product was filtered and washed with dry methanol, and provided a quantitative and pure yield of conjugated the desired pPEI product.

The PEI used to form the conjugate had an average molecular weight of 25K. The produced PEG-LPEI “pPEI” conjugate comprised an average of 10% PEG (mole/mole) and had an average activated mPEG molecular weight of 407, thus denoting a PEG-LPEI “pPEI” conjugate in which each PEG conjugate comprised approximately 9 ethylene glycol monomer substituents (i.e., 407 PEG molecular weight/44 ethylene glycol substituent molecular weight=approximately 9 ethylene glycol monomer substituents per PEG conjugate).

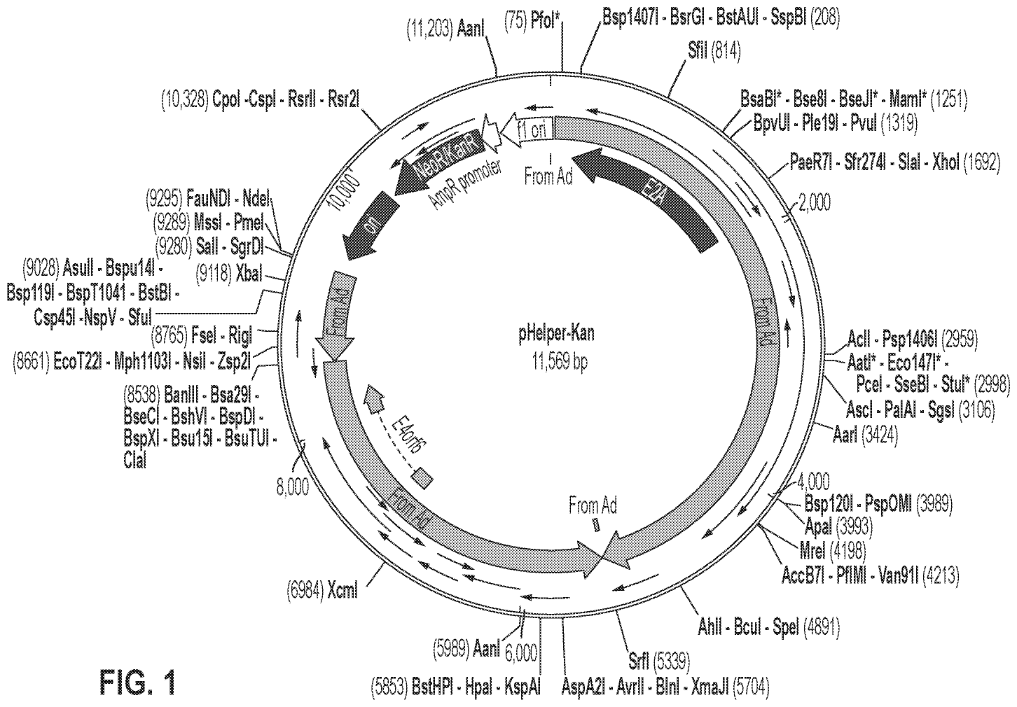

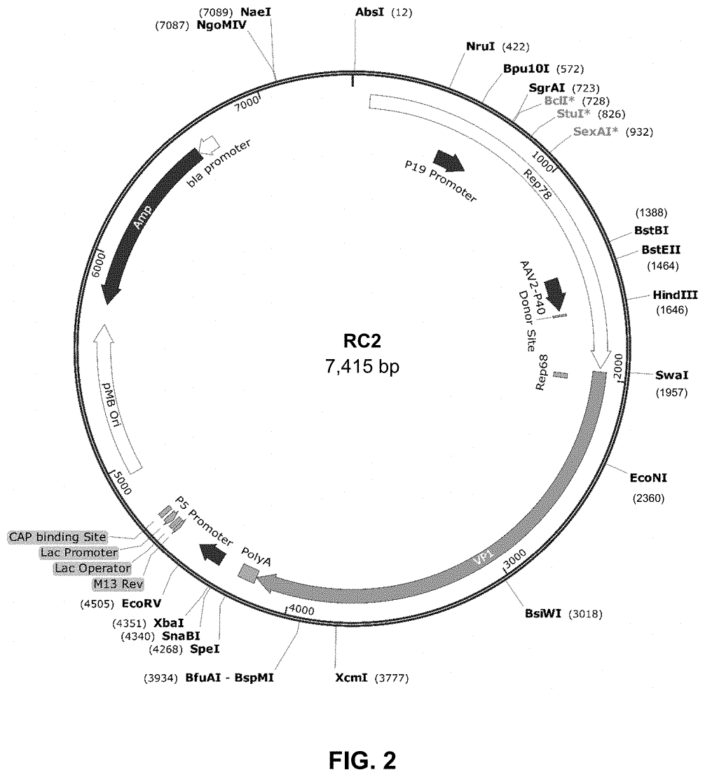

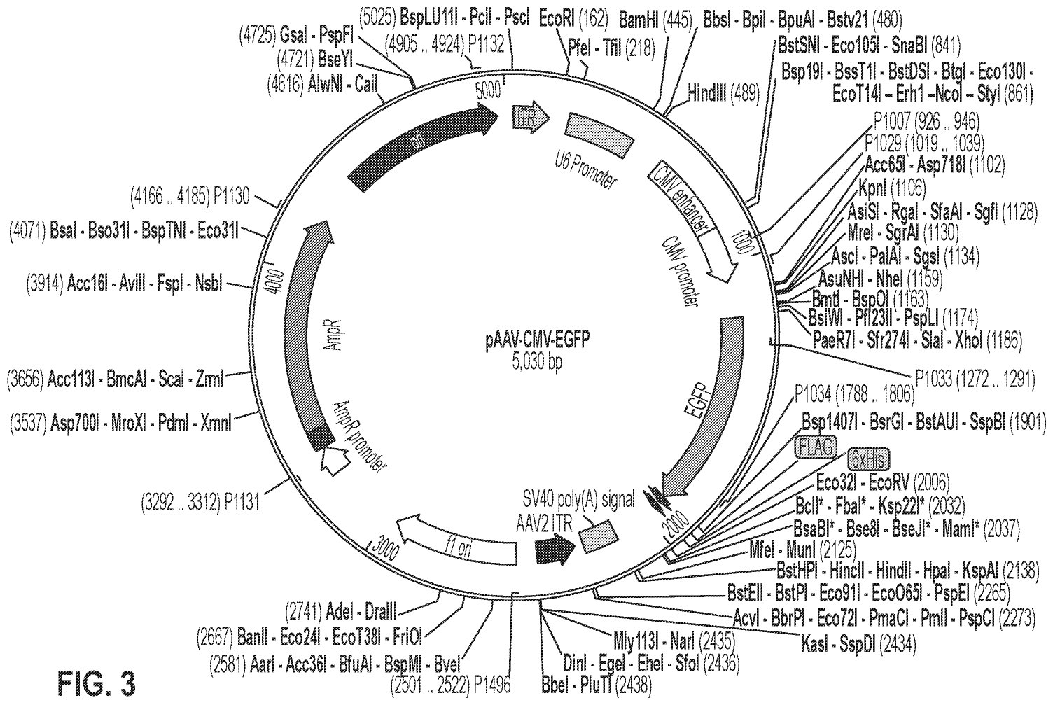

To demonstrate the improved transfection efficiency, 20 ml of HEK293 cells grown in suspension at a density of 2×10⁶ cells/ml) were transfected with the plasmids:
pHelper (also known as pHelper-Kan; U.S. Pat. No. 10,557,149; FIG. 1), which is 11,569 nucleotides in length, and is an illustrative helper plasmid capable of providing the viral transcription and translation factors (E1a, E1b, E2a, VA and E4) required for AAV proliferation;
RC vector (also known as RC2; U.S. Pat. No. 10,557,149; FIG. 2), which is 7,415 nucleotides in length, and is an illustrative plasmid for providing the AAV2 rep and cap genes; and
pAAV2-CMV-GFP (also known as pAV-CMV-EGFP; U.S. Pat. No. 10,557,149; FIG. 3) is 5,030 nucleotides in length, and is an illustrative rAAV plasmid designed to express the enhanced green fluorescent protein (EGFP) (Gambotto, A. et al. (2000) “Immunogenicity Of Enhanced Green Fluorescent Protein (EGFP) In BALB/C Mice: Identification Of An H2-Kd-Restricted CTL Epitope,” Gene Ther. 7(23):2036-2040; Tsien, R. Y. (1998) “The Green Fluorescent Protein,” Annu. Rev. Biochem. 67:509-544; Cinelli, R. A. et al. (2000) “The Enhanced Green Fluorescent Protein As A Tool For The Analysis Of Protein Dynamics And Localization: Local Fluorescence Study At The Single-Molecule Level,” Photochem. Photobiol. 71(6):771-776; Chopra A. (2008) “Recombinant Adenovirus With Enhanced Green Fluorescent Protein,” In: MOLECULAR IMAGING AND CONTRAST AGENT DATABASE (MICAD), National Center for Biotechnology Information, Bethesda MD) under the control of a cytomegalovirus (CMV) promoter and enhancer. The complete nucleotide sequence of the coding strand of pAAV2-CMV-GFP (pAV-CMV-EGFP) is disclosed in U.S. Pat. No. 10,557,149, herein incorporated by reference.

The transfection was conducted in the presence of commercially available PEI transfection reagents that are not PEI-PEG co-polymers: PEIMax® (Polysciences) or PEIPro® (Polyplus), or in the presence of the above-described PEG-LPEI conjugate (“pPEI”) (at a 2:1 or 1.5:1 ratio of reagent to DNA). The ratio of DNA and activated mPEG-LPEI is 1 μg to 4 The extent of transfection was determined by AAV virus production using q-PCR amplification with primers hybridizing to the ITR domain of the produced rAAV (see, e.g., Ahlemeyer, B. et al. (2020) “Analysis of the Level of Plasmid-Derived mRNA in the Presence of Residual Plasmid DNA by Two-Step Quantitative RT-PCR,” Methods Protocols 3:40:1-12; Cohen, R. N. et al. (2009) “Quantification Of Plasmid DNA Copies In The Nucleus After Lipoplex And Polyplex Transfection,” J. Control. Release 135(2):166-174).

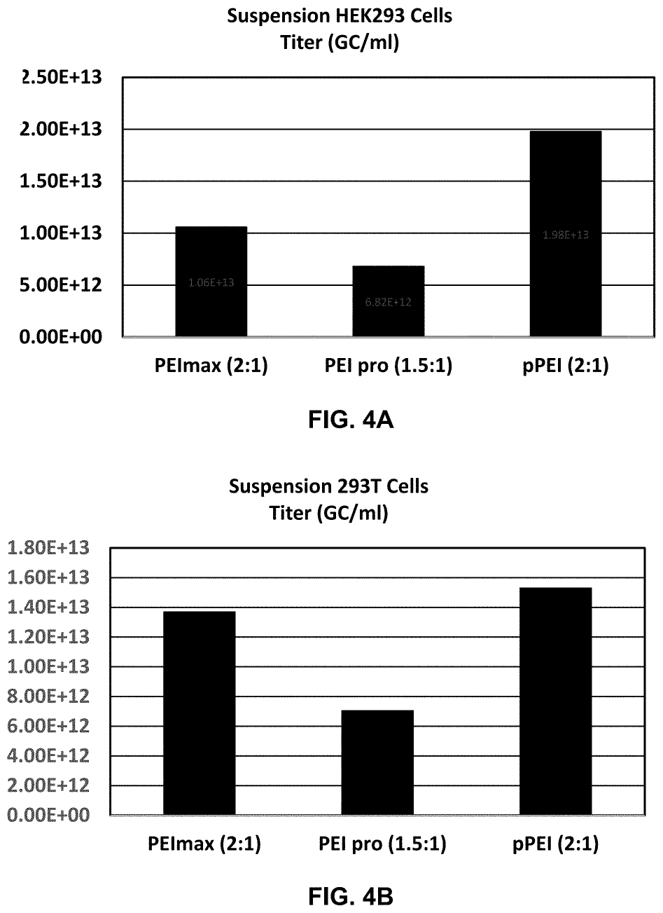

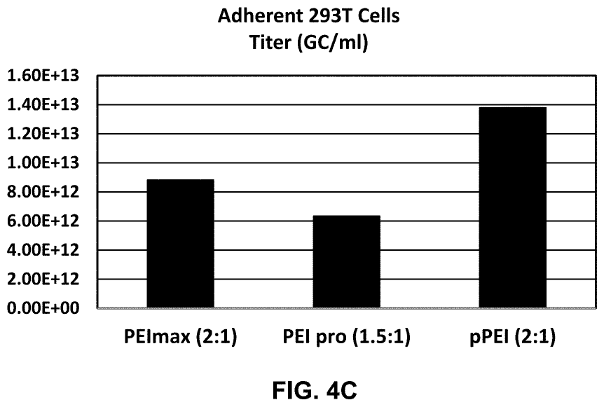

FIGS. 4A-4C show the titers of transfected suspension HEK293 cells (FIG. 4A), suspension 293T cells (FIG. 4B), and adherent 293 T cells (FIG. 4C) found in the crude cell pellets. Titers were measured 48 hours post transfection. The reported titers are averaged from three independent samples 48 hours post transfection As shown in FIGS. 4A-4C, in all cases the use of the above-described PEG-LPEI conjugate (“pPEI”) resulted in a significantly improved transfection efficiency and/or significantly improved viability relative to PEI.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A polyethylene glycol conjugate of linear polyethyleneimine comprising the Formula (III): 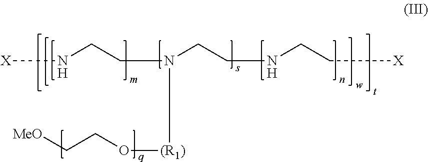
wherein: R₁ is a carbon-containing, hydroxyl-comprising group that comprises one, two, three, or more than three, carbon atoms;
each X is independently amine, methyl or methoxy, or an alternative terminating atom or chemical group;
m and n are independently zero or an integer;
s, w and t are independently an integer equal to or greater than 1;
m, n, and s may be the same, or may vary independently, for each w; and
q is an integer equal to or greater than 1, and may vary independently for each s.

2. The polyethylene glycol conjugate of polyethyleneimine (III) of claim 1, wherein R₁ is —CH₂—CH(OH)—CH₂—, and said polyethylene glycol conjugate of polyethyleneimine (III) is:

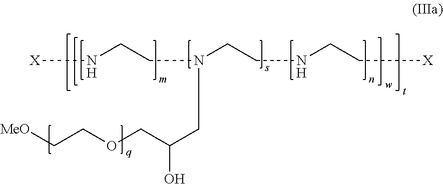

3. The polyethylene glycol conjugate of polyethyleneimine (III) of claim 1, wherein the conjugate comprises from 1 to approximately 2325 ethyleneimine monomer substituents and/or from 1 to approximately 2272 ethylene glycol substituents.

4. (canceled)

5. The polyethylene glycol conjugate of polyethyleneimine (III) of claim 1, wherein about 10% of ethyleneimine monomer substituents of said polyethylene glycol conjugate of polyethyleneimine are conjugated to polyethylene glycol.

6. A method of producing the polyethylene glycol conjugate of polyethyleneimine (III) of claim 1, which comprises:
(A) reacting a polyethyleneimine of Formula (I) 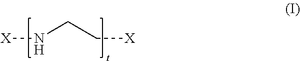
with a polyethylene glycol of Formula (II) 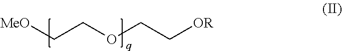
wherein R is an activated leaving group that reacts with a secondary amine N of (I) to form said carbon-containing, hydroxyl-comprising group R₁ that comprises one, two, three, or more than three, carbon atoms,
in MeOH at neutral or basic pH.
(B) acidifying the reaction to thereby cause said polyethylene glycol conjugate of polyethyleneimine (III) to become insoluble; and
(C) recovering said polyethylene glycol conjugate of polyethyleneimine (III) from the insoluble fraction of the reaction.

7. The method of claim 6, wherein said polyethylene glycol (II) is polyethylene glycol-2-(methoxy)oxirane, R₁ is —CH₂—CH(OH)—CH₂—, and said polyethylene glycol conjugate of polyethyleneimine (III) is:

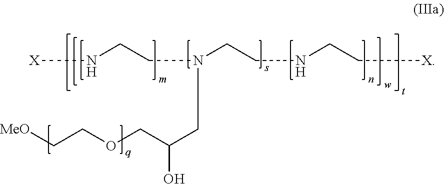

8. A composition comprising the polyethylene glycol conjugate of polyethyleneimine (III) of claim 1 and a polynucleotide molecule.

9. The composition of claim 8, wherein said composition is suitable for transfecting said polynucleotide into a recipient cell, wherein the presence of said polyethylene glycol conjugate of polyethyleneimine (III) enhances the efficiency of transfection or the viability of transfected cells relative to the efficiency of transfection or the viability of transfected cells when transfected in the absence of said polyethylene glycol conjugate of polyethyleneimine (III).

10. The composition of claim 9, wherein said efficiency of transfection or viability in the presence of said polyethylene glycol conjugate of polyethyleneimine (III) is at least 50% greater than the efficiency of transfection or the viability of such transfected cells when transfected in the absence of said polyethylene glycol conjugate of polyethyleneimine (III).

11. A composition comprising the polyethylene glycol conjugate of polyethyleneimine (III) of claim 2 and a polynucleotide molecule.

12. The composition of claim 11, wherein said composition is suitable for transfecting said polynucleotide into a recipient cell, wherein the presence of said polyethylene glycol conjugate of polyethyleneimine (III) enhances the efficiency of transfection or the viability of transfected cells relative to the efficiency of transfection or the viability of transfected cells when transfected in the absence of said polyethylene glycol conjugate of polyethyleneimine (III).

13. The composition of claim 11, wherein said efficiency of transfection or viability in the presence of said polyethylene glycol conjugate of polyethyleneimine (III) is at least 50% greater than the efficiency of transfection or the viability of such transfected cells when transfected in the absence of said polyethylene glycol conjugate of polyethyleneimine (III).

14. The composition of claim 8, wherein said polynucleotide molecule comprises a non-viral vector, optionally wherein the non-viral vector is a plasmid vector.

15. (canceled)

16. The composition of claim 8, wherein said polynucleotide molecule comprises a viral vector, optionally wherein the viral vector is an adeno-associated vector, a lentiviral vector, or an adenoviral vector.

17. (canceled)

18. The composition of claim 8, wherein said composition is a pharmaceutical composition and said polynucleotide molecule encodes a vaccine to a pathogenic bacteria, virus or parasite, optionally wherein the pathogenic bacteria, virus, or parasite comprises M. pneumoniae, S. aureus, influenza, SARS-CoV-2, rubella virus, varicella zoster virus, herpes simplex, herpes zoster, or combinations thereof.

19. (canceled)

20. The composition of claim 8, wherein said composition is a pharmaceutical composition and said polynucleotide molecule is therapeutic for the treatment of a genetic disease, or encodes a protein that is therapeutic for the treatment of a genetic disease.

21. (canceled)

22. A method of treating a disease caused by a pathogenic bacteria, virus or parasite, which comprises administering the composition of claim 18 to a person in need thereof, wherein the polynucleotide molecule of said composition encodes a vaccine to said pathogenic bacteria, virus or parasite.

23. The method of claim 22, wherein said pathogenic bacteria, virus or parasite is selected from one or more of the group consisting of: M. pneumoniae, S. aureus, influenza, SARS-CoV-2, rubella virus, varicella zoster virus, herpes simplex, and herpes zoster.

24. A method of treating a genetic disease which comprises administering the composition of claim 20 to a person in need thereof wherein the polynucleotide molecule of said composition is therapeutic for the treatment of said genetic disease, or encodes a protein that is therapeutic for the treatment of said genetic disease.

25. The method of claim 24, wherein said genetic disease is selected from the group consisting of: achondroplasia, Alzheimer's disease, alpha-1 antitrypsin deficiency, alpha-1 antitrypsin deficiency; antiphospholipid syndrome; attention deficit hyperactivity disorder (ADHD); autism; autosomal dominant polycystic kidney disease; cancer; Charcot-Marie-Tooth Disease; cri du chat syndrome; Crohn's disease, cystic fibrosis, Down syndrome, Duchenne muscular dystrophy; Factor V Leiden and Leiden thrombophilia, familial hypercholesterolemia; fragile X syndrome, Gaucher disease, Hemochromatosis; Hemophilia; Holoprosencephaly; Huntington's disease; multiple sclerosis, Parkinson's disease, phenylketonuria; severe combined immunodeficiency; sickle cell disease; spinal muscular atrophy; Tay-Sachs disease; and thalassemia.
